(19) United States
(12) Patent Application Publication
(10) Pub. No.: US 2011/0136160 A1
(43) Pub. Date: Jun. 9, 2011

(54) ANALYSIS OF GLYCATED PROTEINS

(75) Inventors: Jean-Charles Sanchez, Bernex (CH); Feliciano Priego-Capote, Cordoba (ES)

(73) Assignee: Universite De Geneve, Geneva 4 (CH)

(21) Appl. No.: 13/057,228

(22) PCT Filed: Aug. 21, 2009
(86) PCT No.: PCT/GB2009/051047

§ 371 Date: Feb. 2, 2011

(30) Foreign Application Priority Data
Aug. 28, 2008 (GB) 0815576.4

(51) Int. Cl. G01N 33/68; C12Q 1/37

(52) U.S. Cl. 435/23; 436/87

(57) ABSTRACT

This invention relates to a method for analysis of one or more glycated proteins in a sample, the glycated proteins containing moieties of a natural reducing carbohydrate bound at one or more glycation sites in the proteins, the method comprising: treating the sample with a stable isotopic form of said carbohydrate which is different in mass from the natural carbohydrate, whereby the isotopic form becomes incorporated by glycation in one or more proteins in the sample, and one or more of said proteins are accordingly glycated by the natural reducing carbohydrate and by the isotopic form of the carbohydrate at identical glycation sites; and identifying and/or quantifying the glycated proteins by the difference in mass between the natural carbohydrate and the isotopic form of the carbohydrate at identical glycation sites.

### BACKGROUND OF THE INVENTION

1. Field of the Invention

This invention relates to the analysis of glycated proteins, and more particularly to a method for qualitative and/or quantitative analysis of one or more glycated proteins in a sample.

2. Description of the Related Art

Glycated proteins are formed by non-enzymatic reactions between reducing carbohydrates (e.g. glucose, fructose, ribose) or derivatives (e.g. ascorbic acid etc.) with terminal amino groups or ε amino groups in lysine and arginine residues. This process must be distinguished from that enzymatically catalysed by glycosyl transferase to synthesise glycoproteins involved in many biological processes. Enzymatic glycosylation is based on the attachment of oligosaccharides to specific protein side chains such as asparagine (N-linked), serine and threonine (O-linked), and the C-termini of cell surface proteins (1). Glycosylation is involved in many biological processes in contrast to glycation which is a completely undesired modification from a clinical point of view.

Due to the crucial role of glucose as an energy source in humans, it is the main circulating sugar and, thus, the most relevant molecule in terms of protein glycation. The mechanisms involved in glycation are illustrated in FIG. 5 for glucose as reducing sugar (2). The process starts with the formation of the Schiff base by a condensation reaction between the carbonyl group of the reducing sugar and the amino group of the protein. The next step is the conversion of the thermodynamically unstable Schiff base into the Amadori compound which is considered as the first glycation level. In a second stage, the Amadori compound undergoes a series of dehydration and fragmentation reactions generating a variety of carbonyl compounds such as methylglyoxal, glyoxal, glucosones, deoxyglucosones and dehydroascorbate (3). These are generally more reactive than the original carbohydrate and act as propagators by reactions with free amino groups leading to the formation of a variety of heterogeneous structures irreversibly formed and commonly known as advanced glycation end-products (AGEs). The impact of glycation into the biological context encompasses alterations of the structure, function and turnover of proteins (4). Evidently, the effects of this biological impact will depend on the glycation extent. From a clinical point of view, it would be interesting to detect this post-translational modification (PTM) at the initial stage due to its prognostic and diagnostic applicability.

The kinetics of the initial glycation process are governed by the formation of the Amadori compound which is a slow step under human physiological conditions (37° C., ˜5 mM blood glucose concentration in healthy subjects) (5). However, this process is enhanced under prolonged hyperglycaemia exposition being one of its pathophysio logical mechanisms of action. In contrast to physiological glucose concentration, chronic supraphysio logical glucose concentration (>10 mM) negatively affects a large number of organs and tissues, such as pancreas, eyes, liver, muscles, adipose tissues, brain, heart, kidneys and nerves. Glucose toxicity is the main cause of diabetic complications, which are often observed only several years after the development of the illness (6, 7). However, chronic hyperglycaemia can also increase the development rate of early diabetic states by affecting the secretion capacity of pancreatic cells, which in turn increase blood glucose concentration. This vicious circle finally leads to the total incapacity of β-cells to secrete insulin (8, 9). That is why glycation has often been related to chronic complications of diabetes mellitus, renal failure and degenerative changes occurring in the course of aging (10-12).

Glycation of proteins is one of the potential mechanisms that are expected to be involved in glucotoxicity due to clinical evidences. Calvo et al. have evaluated the non-enzymatic glycation of high-density lipoprotein (HDL) in type 1 and 2 diabetic patients as compared to that on control healthy subjects. The authors isolated glycated apolipoprotein A-I (ApoA-I) from diabetic patients and compared its lipid binding properties to those of ApoA-I from healthy subjects. They found that ApoA-I glycation promotes a decrease in the stability of the lipid-apolipoprotein interaction and also in its self-association. Therefore, the structural cohesion of HDL molecules is seriously affected by glycation of ApoA-I (13-15). In vivo studies in mice proved that glycated insulin exhibits a reduced ability to stimulate glucose oxidation by the isolated mouse diaphragm muscle. This observation was in concordance with previous studies suggesting that glycation of insulin decreases its potency to stimulate lipogenesis in isolated rat adipocytes. Consistent with such effects, glycated insulin displayed a significantly reduced ability to lower plasma glucose concentrations in mice. These and other studies clearly indicated that glycation results in a significant impairment of insulin action to regulate plasma glucose homeostasis (16).

The glycaemic control of clinical patients is currently assessed indirectly with the conventional test for the analysis of glycated haemoglobin (HbA1c). HbA1c is a long-term indicator of the patient glycaemic state because of the erythrocyte lifespan (˜120 days). HbA1c concentration is a memory effect of blood glucose concentrations over the previous 8-12 weeks (17-20). Other measurements indicative of short-term glucose perturbation are needed in order to understand its potential biological effect, taking into account that any protein could be potentially glycated. Due to the continuous exposure to glucose, the concentration of HbA1c and glycated human serum albumin in plasma from healthy patients has been estimated around 5-7% and 15%, respectively (21, 22). Therefore, the development of methods for identification and quantification of glycated proteins as well as for prediction of new potential targets under different conditions is crucial to elucidate their biological effect.

Glycohaemoglobin (HbA1c) is a long term control indicator in patients with diabetes mellitus. The amount of HbA1c reflects the mean glucose concentration over the previous two to three months (lifetime of red blood cells). Known methods for analysis of HbA1c are based on cation exchange chromatography, affinity chromatography and immune turbidimetry. These methods can be interfered with chemical modification of the haemoglobin, e.g. carbamylation or acetylation. Moreover, many conditions alter HbA1c levels. Any process that shortens erythrocyte lifespan (e.g. kidney disease, liver disease, hemolytic anemia, hemoglobinopathies, and recovery from blood loss) decreases HbA1c as glycation increases with age of the red cell. Also lower HbA1c levels are found in diabetic and nondiabetic pregnant women. Any process that slows erythropoesis such as aplastic anemia will increase HbA1c by causing an older erythrocyte cohort. Current methods for measuring HbA1c levels are therefore not useful for prognosis purposes.

U.S. Pat. No. 7,070,948 discloses a method for assaying glycated protein, comprising treating a glycated protein-containing sample with protease to liberate a glycated peptide from a glycated protein; allowing an oxidase to react with the liberated glycated peptide; and determining the produced hydrogen peroxide.

U.S. Pat. No. 7,183,118 discloses methods for quantitative proteome analysis of glycoproteins, involving immobilizing glycopolypeptides to a solid support; cleaving the immobilized glycopolypeptides, thereby releasing non-glycosylated peptides and retaining immobilized glycopeptides; releasing the glycopeptides from the solid support; and analysing the released glycopeptides. The method can include the step of identifying one or more glycopeptides using mass spectrometry. In one embodiment non-enzymatic glycation in diabetic mice is investigated by labelling serum from normal and diabetic obese mice with light and heavy ICAT (isotope coded affinity tag) reagent.

Recently, Zhang et al. proposed several approaches for the characterization of glycated proteins (23-25). These approaches are based on bottom-up workflows characterized by the implementation of selective and sensitive steps for this application such as an enrichment step for isolation of glycated proteins and/or peptides with boronate affinity chromatography (BAC) and data-dependent mass spectrometry methods. For example, the first of these references describes proteomic profiling of non-enzymatically glycated proteins in human plasma and erythrocyte membranes. Phenylboronate affinity chromatography was used to enrich glycated proteins and glycated tryptic peptides. The enriched peptides were subsequently analysed by liquid chromatography coupled with electron transfer dissociation-tandem mass spectrometry for identification of proteins which have been glycated. Nevertheless, these approaches have been focused on qualitative analysis by identification of glycated proteins and sugar attachment sites. Therefore, it is clear that there is a demand for quantitative methods for analysis of glycated proteins in order to evaluate the glycaemic control of clinical samples or compare patient glycaemic states.

A problem with the above proposals is that they do not enable the qualitative and quantitative analysis of complex mixtures of glycated proteins such as those which occur in the human or animal body. In the present invention we have provided methods which enable such analysis, permitting the identification of glycated proteins, identification of glycation sites within the protein structures, and quantitative assay of the degree of glycation at such sites.

### SUMMARY OF THE INVENTION

The present invention provides the following:

1. A method for analysis of one or more glycated proteins in a sample, the glycated proteins containing moieties of a natural reducing carbohydrate bound at one or more glycation sites in the proteins, the method comprising:
treating the sample with a stable isotopic form of said carbohydrate which is different in mass from the natural carbohydrate, whereby the isotopic form becomes incorporated by glycation in one or more proteins in the sample, and one or more of said proteins are accordingly glycated by the natural reducing carbohydrate and by the isotopic form of the carbohydrate at identical glycation sites; and
identifying and/or quantifying the glycated proteins by the difference in mass between the natural carbohydrate and the isotopic form of the carbohydrate at identical glycation sites.

2. A method for analysis of one or more glycated proteins in a sample, the glycated proteins containing moieties of a natural reducing carbohydrate bound at one or more glycation sites in the proteins, the method comprising:

(a) treating the sample with a stable isotopic form of said carbohydrate which is different in mass from the natural carbohydrate, whereby the isotopic form becomes incorporated by glycation in one or more proteins in the sample;
(b) digesting the proteins in the thus-treated sample to form peptides, at least some of which are glycated by the natural reducing carbohydrate and some by the isotopic form of the carbohydrate at identical glycation sites;
(c) separating the glycated peptides from the non-glycated peptides; and
(d) identifying and/or quantifying the glycated peptides by the difference in mass between the natural carbohydrate and the isotopic form of the carbohydrate at identical glycation sites.

3. A method according to 1 or 2, in which the natural reducing carbohydrate is selected from glucose, fructose, ribose, mannose, ascorbic acid, glyoxal and methylglyoxal.

4. A method according to 3, in which the natural reducing carbohydrate is glucose.

5. A method according to any of 1 to 4, in which the isotopic form of the carbohydrate is the 13C isotope, the 2H isotope or the 18O isotope, preferably the 13C isotope.

6. A method according to 5, in which the natural reducing carbohydrate is 12C6-glucose and the isotopic form is 13C6-glucose.

7. A method according to any of 2 to 6, in which the proteins are digested to form peptides by treatment with an endoproteinase, such as Glu-C, trypsin, Asp-N or Arg-C, or with CNBr.

8. A method according to 7, in which the digestion step is carried out with endoproteinase Glu-C.

9. A method according to any of 2 to 8, in which the glycated peptides are separated from the non-glycated peptides by boronate affinity chromatography, cationic exchange chromatography, isoelectric focusing or reverse phase hplc, preferably by boronate affinity chromatography.

10. A method according to any of 1 to 9, in which the glycated proteins or peptides are identified and/or quantified by mass spectrometry, wherein doublet signals are obtained, corresponding to each glycation site, with a mass shift corresponding to the difference in mass between the natural carbohydrate and the isotopic carbohydrate.

11. A method according to 10, in which the glycated proteins or peptides are quantified by measuring the signal intensity corresponding to glycation with the natural carbohydrate and comparing it to the signal intensity corresponding to glycation with a predetermined quantity of the isotopic form of the carbohydrate at the same glycation site.

12. A method according to 10 or 11, in which tandem mass spectrometry is carried out to identify and/or quantify the glycated peptides, and hence identify proteins from which the glycated peptides have been derived and the glycation sites for a specific protein in the sample, and optionally to quantify the degree of glycation at such sites.

13. A method according to any of 10 to 12, in which the glycated peptides are fractionated by reversed-phase liquid chromatography prior to analysis by mass spectrometry.

### BRIEF DESCRIPTION OF THE FIGURES

FIG. 1 is a diagrammatic representation of the analytical workflow for qualitative and quantitative analysis of glycated proteins in an embodiment of the invention;

FIG. 2 is a diagrammatic representation of the mechanism involved in the separation of glycated and non-glycated peptides by boronate affinity chromatography;

FIG. 3 comprises mass spectrometry data showing the detection of peptides labelled with “light” and “heavy” isotopic glucoses;

FIG. 4 comprises mass spectrometry data providing a comparison between two MS (MS² and MS³) methodologies for one of the glycated peptides identified for the standard tested in FIG. 3 (RGFFYTPK*A from insulin);

FIG. 5 is a scheme of the glycation process;

FIG. 6 is a diagrammatic representation of the bottom-up proteomics workflow for quantitative analysis of glycated proteins in an embodiment of the invention;

FIG. 7 shows different activation modes in mass spectrometry analysis of glycated proteins from human serum albumin;

FIG. 8 shows extracted ion chromatograms in mass spectrometry of immonium ions calculated for glycated lysine in plasma analysis;

FIG. 9 shows mass spectrometry data for a human serum albumin glycated peptide identified in plasma;

FIG. 10 is a mass scan showing two glycated peptides from horse myoglobin and bovine insulin;

FIG. 11 shows MS precursor scans of glycated peptides containing the five preferential glycation sites in human serum albumin;

FIG. 12 shows glycation affinity of the different sites identified in four glycated proteins found in plasma;

FIG. 13 shows a scatterplot for the SuperHirn and manually determined ratios between peak areas provided by the in vivo and in vitro labelled peptides for an experiment assessing the real glycaemic state (Example 5), and a scatterplot of the log-intensities of the light and heavy features;

FIG. 14 shows MS spectra of a human serum albumin glycated peptide identified in analysis of plasma, and the number of glycated peptides identified in plasma vs charge state of these identifications.

### DETAILED DESCRIPTION

Non-enzymatic glycation of proteins is a post-translational modification produced by a reaction between reducing sugars and amino groups located in lysine and arginine residues or in N-terminal position. This modification plays a relevant role in medicine and food industry. In the clinical field, this undesired role is directly linked to blood glucose concentration and, therefore, to pathological conditions derived from hyperglycaemia (>11 mM glucose) such as diabetes mellitus or renal failure. An approach for qualitative and quantitative analysis of glycated proteins is here described to achieve the three information levels for their complete characterization. These are identification of glycated proteins, elucidation of sugar attachment sites and quantitative analysis to compare between glycaemic states. In embodiments of the invention, qualitative analysis can be carried out by tandem mass spectrometry after endoproteinase Glu-C digestion and boronate affinity chromatography for isolation of glycated peptides. For this purpose, two MS operational modes can be used: HCD-MS2 and CID-MS3 by neutral loss scan monitoring of two selective neutral losses (162.05 and 84.04 Da for the glucose cleavage and an intermediate rearrangement of the glucose moiety). On the other hand, quantitative analysis can be based on labelling of proteins with ¹³C₆-glucose incubation in order to evaluate the native glycated proteins labelled with ¹²C₆-glucose. As glycation is chemo-selective, it is exclusively occurring in potential targets for in vivo modifications. This approach, named Glycation Isotopic Labelling (GIL), implemented on a bottom-up workflow enabled to differentiate glycated peptides labelled with both isotopic forms resulting from enzymatic digestion by mass spectrometry (6 Da mass shift/glycation site). The strategy was then applied to a reference plasma sample that allowed detection of 50 glycated proteins and 161 sugar attachment positions with identification of preferential glycation sites for each protein. A predictive approach was also tested to detect potential glycation sites under high glucose concentration.

An innovative method for quantitative analysis of glycated proteins is here presented. In one embodiment, this method is based on differential labelling of proteins with isotopic [¹³C]-sugars, named Glycation Isotopic Labelling (GIL). The labelling step is performed by natural incubation under physiological conditions mimicking the in vivo glycation process. By this procedure, only potential glycation targets are labelled due to the chemo-selectivity of this process. After labelling, this approach can be implemented to any proteomics workflow based on MS detection as the two isotopic forms of a glycated protein can be discriminated. In this research, the approach has been implemented in a bottom-up workflow for analysis of non-enzymatic glycation of the human plasma proteome. The data analysis can be fully automated and has been performed combining Phenyx MS2 identification (26ref) and SuperHirn MS1 quantification tools (27ref).

The present invention provides a qualitative and quantitative method to assess glycemic control at short and long-term exposure to high glucose concentrations. In contrast to HbA1c analysis methods our alternative is focused on the analysis of the full proteome of the target sample (blood, plasma/serum or other biological fluids). We thus provide an analytical platform to achieve different information levels which are not only quantitative but also qualitative. Our method involves the chemical incorporation of stable isotopes (not affecting amino acids).

As an embodiment of the invention, a semiquantitative approach to analysis of glycated proteins is described. The glycation in this embodiment is with glucose as the natural reducing carbohydrate. Two aliquots of a target sample are incubated for equal or different times with equal or different quantities of, respectively, “light” glucose (in which all six carbon atoms are 12C, also referred to as 12Glu6) and “heavy” glucose (in which all six carbon atoms are 13C, also referred to as 13Glu6). The incubated samples are pooled and digested with a suitable enzyme such as endoproteinase Glu-C. The resulting peptides are separated into non-glycated peptides and glycated peptides by boronate affinity chromatography. Reversed phase fractionation of the peptides is carried out, followed by tandem mass spectrometry and data analysis. The MS data is plotted as abundance (%) against mass/charge ratio (m/z). Glycated peptides are identified by doublet signals separated by a 6 Dalton mass shift per glycation site (or fraction of 6 if the peptide is more than singly charged). This procedure achieves the following:
Identification of target proteins for potential glycation according to the exposure time to glucose concentrations above the physiological range.
Distinction between proteins to be glycated at short and long-term periods.
Identification of glycation peptides for absolute quantification of the target proteins.
Identification of glycation sites which is of particular interest for elucidation of their biological effect.
This semiquantitative approach enables comparison between two glycation states for the same sample.

An absolute quantitative approach can then be adopted as follows. Once target glycated peptides have been identified, there are two strategies to be followed for the assessment of the glycemic control by absolute quantification:

1) spiking a control sample (red blood cells) incubated with 13Glu6 to a test sample at different ratios;
2) spiking target glycated peptides labeled with 13Glu6 (obtained by absolute quantification synthesis, AQUA) to a test sample at different ratios.

As the mass of the different peptides is defined, this analysis can be carried out by LC-MS/MS by multiple reaction monitoring (MRM) which enables the highly selective and sensitive determination of target glycated proteins. Glycated peptides with 13Glu6 act as internal standards. This methodology is useful for clinical prognosis of irregularities in glycemic control caused by exposure to high glucose concentrations. An absolute quantification approach is for the first time proposed for analysis of glycated proteins.

The following Examples illustrate the invention.

### LIST OF ABBREVIATIONS AND SYMBOLS

Abbrev. Definition
AGEs Advanced glycation end-products
ApoA-I Apo lipoprotein A-I
BAC Boronate affinity chromatography
CID Collision-induced dissociation
ETD Electron transfer dissociation
FDR False discovery rate
GIL Glycation isotopic labelling
HAc Acetic acid
HbA1c Glycated haemoglobin
HbSAg Hepatitis B surface antigen
HCD High energy collisional dissociation
HCV Hepatitis C virus
HDL High density lipoprotein
HSA Human serum albumin
IAA Iodoacetamide
PTM Post-translational modification
TCEP Tris-(2-carboxyethyl)-phosphine hydrochloride
TEAB Triethylammonium hydrogen carbonate buffer

### Example 1

### Quantitative Analysis of Glycated Proteins

This example describes the strategy for purification of glycated peptides, relative quantitation by labelling with stable isotopes and identification of glycated peptides.

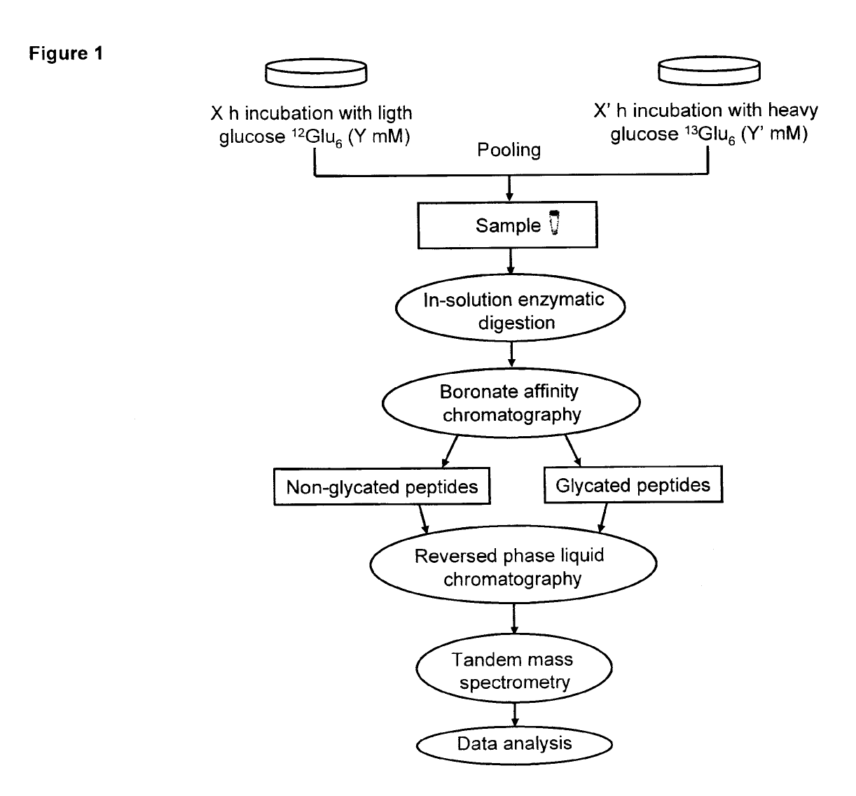

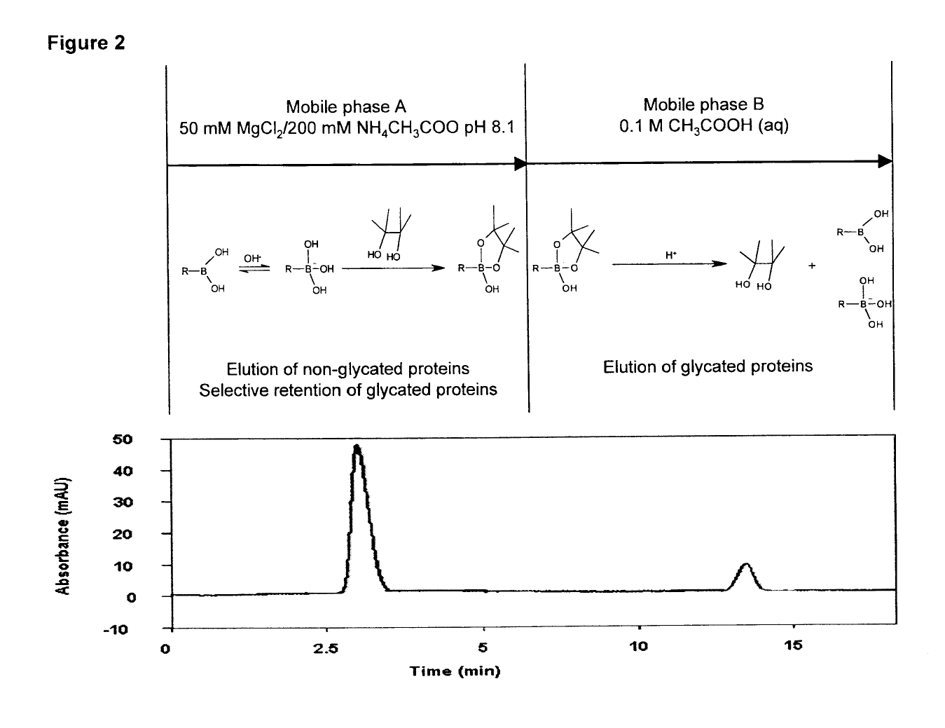

A scheme of this quantitative approach is illustrated in FIG. 1 which shows the different steps of the analytical workflow to be followed. These are: (1) Separated incubation of two aliquots of a test sample with “light” and “heavy” isotopic glucose (defining “light” glucose as that in which all carbons correspond to the isotope ¹²C, whereas in “heavy” glucose all carbons correspond to the isotope ¹³C); (2) Pooling of both incubation sets; (3) In-solution enzymatic digestion for peptides generation; (4) Separation of glycated and non-glycated peptides by boronate affinity chromatography (see FIG. 2); (5) Reversed-phase liquid chromatography (RPLC); (6) Tandem mass spectrometry analysis; and, finally, (7) Analysis of the data sets by a suite of software tools.

Four conventional proteins (myoglobin, β-lactoglobulin, insulin and lysozyme) were dissolved (100 nanomols of each one) in a buffer containing 100 mM NaH₂PO₄, pH 7.5 (incubation buffer). The standard was split into two aliquots which were incubated separately with 90 mM “light” and “heavy” glucose for 24 hours at 37° C. in order to simulate physiological conditions. Then, both sets were pooled to subject the resulting mixture to the rest of the analytical workflow. This is initiated by desalting and isolation of proteins using centrifugal filtration units (Microcon with cut-off 3 kDa as nominal molecular weight limit). Proteins were reconstituted in 0.5 M triethylammonium hydrogen carbonate buffer (TEAB, digestion buffer), pH 8.5 and split into five sub-samples (400 μL each one) for enzymatic digestion. The protocol for digestion of one sub-sample started with the addition of 20 μL of 50 mM tris-(2-carboxyethyl) phosphine hydrochloride (TCEP) for 60 min at 60° C. in order to reduce disulfide bonds. Then, iodoacetamide (IAA) at a concentration of 400 mM was added (10 μL) to alkylate thiol groups. The mixture was reacted for 30 min in the dark at room temperature. Freshly prepared endoproteinase Glu-C (1 μg/μL) was added (100 μL to obtain a 1:10 w/w ratio), and the digestion was performed overnight at 37° C. After protein digestion, the resulting solution was dried by evaporation in a speed-vac concentrator and reconstituted in 100 μL of a solution 50 mM MgCl₂/200 mM NH₄CH₃COO at pH 8.1 (adjusted with diluted NaOH). At this moment, this solution contains a mixture of non-glycated and glycated peptides (labelled with “light” and “heavy” glucose).

Following with the proposed workflow, the next step is the separation of glycated peptides from the non-glycated ones. This can be selectively carried out by boronate affinity chromatography (see FIG. 2). This technique is based on the interaction between boronate stationary phase and cis-diol groups (present in the glucose molecule) by esterification under alkaline conditions. This was the chromatographic method used:
Chromatographic column: TSKgel Boronate-5PW (Sigma-Aldrich) with 10 μm particle size.
Flow-rate: 0.7 ml/min.
Room temperature.
Chromatographic mobile phase A: 50 mM MgCl₂/200 mM NH₄CH₃COO at pH 8.1 (adjusted with diluted NaOH).
Chromatographic mobile phase B: 0.1 M CH₃COOH.
Chromatographic method: (1) 100% mobile phase A for 10 min for elution of the non-glycated peptides while the glycated ones are retained; (2) 100% mobile phase B for 10 min for elution of the glycated peptides. The column is equilibrated with mobile phase A for 5 min between analyses.
Sample injection volume: 100 μL.

With this chromatographic method, selective separation of glycated and non-glycated peptides can be achieved. Both peptide fractions are collected and dried in a speed-vac concentrator. The resulting residue is reconstituted in 1 mL 0.1% trifluoroacetic acid (TFA)/5% CH₃CN (v/v) in water for desalting of peptides by using solid-phase extraction cartridges (SPE, Waters Oasis HLB 10 mg cartridges). The SPE protocol consisted of the following steps: (1) Wash of cartridges with 1 ml 0.1% TFA/95% CH₃CN in water (twice); (2) Equilibration of cartridges with 1 ml 0.1% TFA/5% CH₃CN in water (twice); (3) Addition of sample solution; (4) Wash of cartridges with 1 ml 0.1% TFA/5% CH₃CN in water (twice); and (5) Elution of peptides from cartridges with 1 ml 0.1% TFA/50% CH₃CN in water. With this protocol, desalting of peptides is ensured as well as removal of polar compounds. The eluted solution is dried and peptides reconstituted with 0.1% TFA (aq) for subsequent analysis by RPLC-MS/MS with an electrospray interface (ESI) as ionization source. The separation was run for 60 min using a gradient from 0.1% TFA/3% CH₃CN in water (mobile phase A) to 0.085% TFA/95% CH₃CN in water (mobile phase B). The gradient was run as follows: 0-10 min 100% A, then to 90% A and 10% B at 12 min, 50% A and B at 55 min, and 98% B at 60 min at 400 nL/min flow rate.

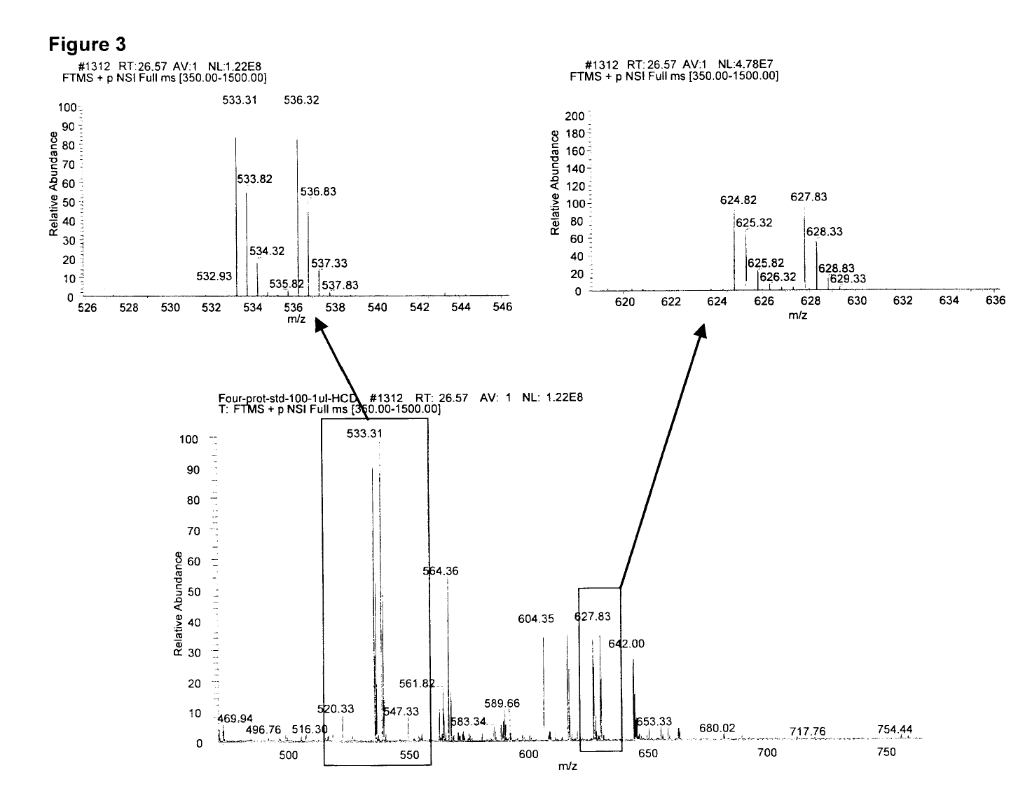

The labelling with both isotopic glucoses enables the detection of glycated peptides by mass spectrometry as they provide a doublet signal with a mass shift of +6 Da (for singly charged peptides, while it would be +3 Da and +2 Da for doubly and triply charged peptides according to the mass/charge ratio, m/z) per glycation site. FIG. 3 shows an example about this detection capability obtained by RPLC-MS in which two glycated peptides are co-eluting. This example was obtained by incubation of the standard composed of four reference proteins with a ratio between “light” and “heavy” glucose equal to 1. The mass spectrum is obtained by RPLC-MS analysis of the four proteins standard (retention time 26.57 min) in which two glycated peptides co-elute (doubly charged peptides with 533.31 and 624.82 m/z for the “light” forms). As can be seen, the intensity of MS signals corresponding to the two versions of the peptide labelled with both isotopic glucose forms is the same. The doublet signals are at 533.31/536.32 and 624.82/627.83, in each case corresponding to a mass shift of 3 Da, as both peptides are doubly charged. This proves that both forms of glucose possess similar glycation efficiency which is a critical aspect for the implementation of isotopic glucose labelling as a quantitative approach. Thus, the isotopic glucose labelling is valid as a quantitative approach to compare between two glycation states for the same sample. This can be performed by measuring the ratio between the MS intensity signals of glycated peptides labelled with “light” and “heavy” glucose.

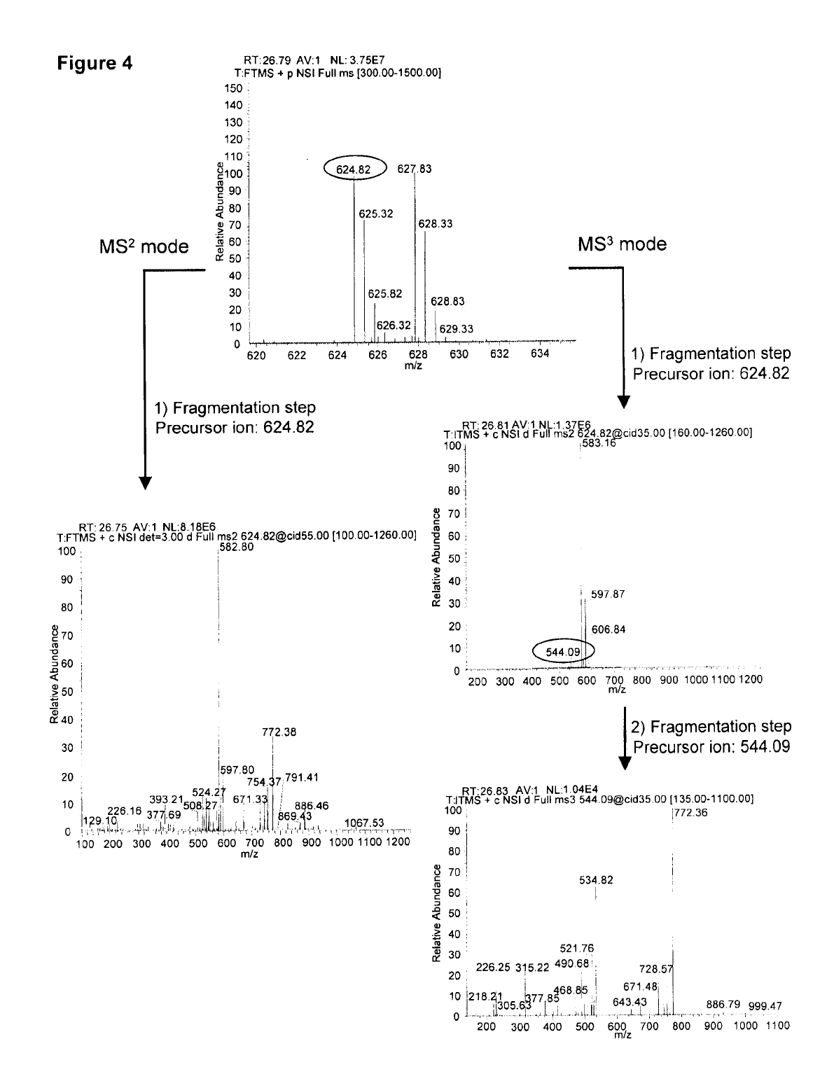

There is no sense in the development of quantitative approaches if this is not supported by qualitative tools focused on the identification of glycated peptides. Tandem mass spectrometry (after RPLC) is an effective tool in this task together with the use of MS/MS fingerprinting identification software (such as Phenyx from Genebio or Mascot from Matrix Science). These software are powerful search engines that use MS and MS/MS data for identification of peptides and proteins from primary sequence databases. In the case of glycation with glucose, the identification is carried out by search of peptides with the addition of 162.0528 mass units (in the mass of the peptide, but also by analysis of its fragmentation pattern in MS/MS). The figure of 162.0528 is derived from the mass of the glucose molecule less the mass of the water molecule which is lost in binding. The identification was carried out by application of two MS/MS methodologies: MS/MS in high collision dissociation energy (MS²) and MS/MS/MS by neutral loss scan (MS³). FIG. 4 shows the operation mode of both methodologies with one of the glycated peptides identified for the standard tested (RGFFYTPK*A from insulin, where K* indicates glycated lysine). In MS² mode, each signal obtained by MS scanning corresponds to a peptide (624.82 m/z that fits with the doubly charged peptide) that is fragmented resulting in a MS/MS spectrum. This is a fingerprinting specific for this peptide and key for its identification. The MS³ mode is similarly initiated with a MS scanning step providing the mass of peptides contained in a test sample. Then, a first fragmentation is carried out by application of a low value of collision energy in order to promote the cleavage of the glucose moiety (neutral loss of 162.0528 mass units). A neutral loss of 162.0528 mass units corresponds to a loss of half this value for a doubly charged peptide, and a peptide of 544.09 m/z is selected for further fragmentation. The peptide in which the neutral loss is detected, i.e. 544.09 m/z, is physically isolated for the second fragmentation with a standard collision energy value. The MS/MS spectrum obtained in the second fragmentation provides the sequence of the glycated peptide after removal of the glucose moiety. Therefore, the MS3 mode is a more selective step as only those ions losing the mass corresponding to the glucose moiety are isolated for a second fragmentation step.

An additional level of information is achieved by identification of the glycation sites. The elucidation of the position where glycation takes place is of particular interest to interpret its biological effect on the protein function or turnover. It is possible to identify the glycation site with both MS/MS methodologies. Thus, in MS² mode the spectra obtained by fragmentation of glycated peptides contain this information that is processed with the fingerprinting software. Concerning the MS³ mode, the data treated for sequencing of the peptides are those obtained in the second fragmentation step. As the glucose moiety is removed in the first MS/MS step, it would not be possible to identify the glycation site. However, if a second neutral loss is simultaneously monitored (loss of 84 mass units by intermediate fragmentation of the glucose moiety), it is possible to know where the glucose was attached. Table 1 shows the glycated peptides together with the attachment sites that were identified with both MS/MS methodologies by analysis of the four proteins standard.

TABLE 1
Glycated peptides identified with both
MS/MS methodologies for analysed proteins

| | MS² mode | MS³ mode |
|---|---|---|
| Horse_myoglobin | KFDKFKHLKTEAE | KFDKFKHLKTEAE |
| | MKASEDLKKHGTVVLT | MKASEDLKKHGTVVLT |
| | SHATKHKIPIKYLE | SHATKHKIPIKYLE |
| | SHATKHKIPIKYLE | SHATKHKIPIKYLE |
| | LFRNDIAAKYKE | |
| | ALGGILKKKGHHEAE | |
| | NVWGKVEADIAGHGQE | |
| Bovine_β- | NKVLVLDTDYKKY | NKVLVLDTDYKKY |
| lactoglobulin | KFDKALKALPM | KFDKALKALPM |
| Bovine_Insulin | RGFFYTPKA | RGFFYTPKA |

| | |
|---|---|
| FVNQHLCGSHLVE | FVNQHLCGSHLVE |

K: Glycated lysine;
F: N-terminal glycation;
M: Oxidized methionin;
C: Carbamidomethylated cysteine.

Alternatively, this approach can be applied with other reducing carbohydrates (fructose, ribose, mannose, . . . ) or derivatives (ascorbic acid, glyoxal, methylglyoxal, . . . ) by using their “light” and “heavy” forms. In addition the whole analysis could be performed without digestion of the full protein.

This example describes the strategy for purification of glycated peptides, relative quantitation by labelling with stable isotopes and identification of glycated peptides.

### Example 2

### Discovery of Glycated Proteins

This example describes the strategy for discovering and measuring the level of new glycated proteins by spiking a reference protein material (red blood cell lysate, plasma and others) labeled with 13Glu6 to the corresponding patient sample.

The different steps of the analytical workflow to be followed are: (1) Incubation of a reference protein material such as plasma or red blood cell lysate with “heavy” isotopic glucose (defining “heavy” glucose where all carbons correspond to the isotope ¹³C); (2) spiking the sample of interest from patients (red blood cell lysate, plasma, others) with the corresponding heavy labelled reference protein material; (3) In-solution enzymatic digestion for peptides generation; (4) Separation of glycated and non-glycated peptides by boronate affinity chromatography; (5) As described in Example 1, analysis of the glycated fraction by reversed-phase liquid chromatography (RPLC), tandem mass spectrometry, and, finally, analysis of the data sets by a suite of software tools.

### Example 3

### Quantitative Analysis of Glycated HbA1c Levels

The level of glycohemoglobin is increased in the red blood cells of persons with poorly controlled diabetes mellitus. Since the glucose stays attached to hemoglobin for the life of the red blood cell (normally about 120 days), the level of glycohemoglobin reflects the average blood glucose level over the past 3 months.

This example describes the strategy for measuring the level of Glycohaemoglobin (HbA1c) levels by spiking the N-terminal peptide of Haemoglobin B chain (obtained by absolute quantification synthesis, AQUA) labeled with 13Glu6.

The different steps of the analytical workflow to be followed are: (1) Incubation of the chemically-synthesised N-terminal peptide of Haemoglobin B chain with “heavy” isotopic glucose (defining “heavy” glucose where all carbons correspond to the isotope ¹³C); (2) spiking the sample of interest (red blood cell lysate, plasma, others) with the heavy labelled N-terminal peptide of Haemoglobin B chain; (3) In-solution enzymatic digestion for peptides generation; (4) Separation of glycated and non-glycated peptides by boronate affinity chromatography; (5) Analysis of the glycated fraction by reversed-phase liquid chromatography (RPLC); (6) Tandem mass spectrometry analysis including multiple reaction monitoring (MRM) for selective quantitation; and, finally, (7) Analysis of the data sets by a suite of software tools.

### Example 4

### Quantitative Analysis of a Panel of Glycated Proteins

The level of glycated proteins is increased in the red blood cells and plasma of persons with poorly controlled diabetes mellitus. Since the glucose stays attached to proteins for their life, the level of glycated proteins reflects the average blood glucose level over the past days, weeks and months according to the half-life of each of the proteins.

This example describes the strategy for measuring the level of newly discovered glycated proteins from Example 2 by spiking their glycated peptide (obtained by absolute quantification synthesis, AQUA) labeled with 13Glu6.

The different steps of the analytical workflow to be followed are: (1) Incubation of the chemically-synthesised peptides of the newly discovered glycated proteins with “heavy” isotopic glucose (defining “heavy” glucose where all carbons correspond to the isotope ¹³C); (2) spiking the sample of interest (red blood cell lysate, plasma, others) with the heavy labelled peptides of the newly discovered glycated proteins; (3) In-solution enzymatic digestion for peptides generation; (4) Separation of glycated and non-glycated peptides by boronate affinity chromatography; (5) Analysis of the glycated fraction by reversed-phase liquid chromatography (RPLC); (6) Tandem mass spectrometry analysis including multiple reaction monitoring (MRM) for selective quantitation of each spiked peptide; and, finally, (7) Analysis of the data sets by a suite of software tools.

### Example 5

### Experimental Procedures

Chemicals—Disodium hydrogen phosphate, sodium hydroxide, ammonium acetate, acetic acid, [¹²C₆]-glucose (≧99.5%) and [¹³C₆]-glucose (99 atom % ¹³C) were purchased from Sigma. Myoglobin from horse heart (≧90%), β-lactoglobulin from bovine milk (˜90%) and insulin from bovine pancreas (powder cell culture tested) were provided by Sigma. Lysozyme from hen egg white (10 500 units mg-1) was from Fluka. These four proteins were used to prepare a multistandard mix in 0.1 M phosphate buffer pH 7.5. Human reference plasma containing 3.8% trisodium citrate as anticoagulant was purchased from Sigma. Plasma was tested and found negative for antibody to HIV-1/HIV-2, antibody to HCV and HbSAg. According to manufacturer, whole blood was collected with anticoagulants (9:1), pooled and centrifuged. The resulting plasma was filtered (0.45 μm) and lyophilized. Triethylammonium hydrogen carbonate buffer (TEAB, 1 M pH 8.5), iodoacetamide (IAA, ≧99%), tris-(2-carboxyethyl) phosphine hydrochloride (TCEP, 0.5 M) and sodium phosphate were from Sigma-Aldrich. Endoproteinase Glu-C from Staphylococcus aureus V8 was from Fluka. Water for chromatography LiChrosolv and acetonitrile Chromasolv for HPLC (≧99.9%) were, respectively, from Merck and Sigma. Superpure ULC-MS formic acid (≧99.9%) was purchased from Biosolve Chemicals (Valkenswaard, the Netherlands) as ionizing agent for LC-MS analysis.

Glucose labelling of a proteins multistandard—Two aliquots of the multistandard of four model proteins (0.125 mg of each protein) in 0.5 ml phosphate buffer were independently incubated with 30 mM [¹²C₆]-glucose and [¹³C₆]-glucose for 24 h at 37° C. Glucose and other salts were removed with Microcon ultrafiltration devices that have an Ultracel® YM-3 regenerated cellulose membrane with 3 kDa molecular weight cut-off (Millipore), followed by a buffer exchange to 0.5 M pH 8.5 TEAB in the same unit according to the manufacturer's instructions. Protein concentration was subsequently measured using the Bradford assay with bovine serum albumin as calibration protein.

Glucose labelling of the reference human plasma—Human plasma was reconstituted in 5 ml water according to the recommended manufacturer protocol. Two aliquots of the reconstituted plasma (50 μl each) in 0.5 ml phosphate buffer were independently incubated with 30 mM [¹²C₆]-glucose and [¹³C₆]-glucose for 24 h at 37° C. Then, each aliquot was separately analysed or were pooled in 1:1 ratio, depending on the analytical purpose, for subsequent analysis with a bottom-up approach. In any case, glucose and other salts were similarly removed by Microcon devices in order to isolate the proteins that were reconstituted in 0.5 M pH 8.5 TEAB. Protein concentration was subsequently measured using the Bradford assay with bovine serum albumin as calibration protein.

Endoproteinase Glu-C enzymatic digestion of proteins—Reconstituted proteins in the case of the multistandard (400 μl) and 1 mg plasma proteins according to Bradford assay (diluted to 400 μl TEAB) were enzymatically digested using endoproteinase Glu-C. For this purpose, cysteine groups were reduced with 50 mM TCEP in water (20 μl) by incubation of the reaction mixtures for 60 min at 60° C. Then, cysteine residues were alkylated with 400 mM IAA (10 μl) for 30 min in the dark at room temperature. Freshly prepared endoproteinase Glu-C (1.0 μg/μl) was added (67 μl to obtain a ratio 1:15 w/w), and the digestion was performed overnight at 37° C. Then, digestion mixtures were evaporated under speed-vacuum and reconstituted in 50 μl mobile phase A (0.2 M NH₄Ac/50 mM MgCl₂ pH 8.1) for isolation of glycated peptides.

Enrichment of glycated peptides by boronate affinity chromatography—Reconstituted peptides were fractioned by boronate affinity chromatography for isolation of the low-concentrated glycated peptides. For this purpose, the target sample (50 μl) was injected in a Waters HPLC equipped with a TSK-Gel boronate affinity column Tosoh Bioscience (7.5 cm×7.5 mm inner diameter; 10 μm particle size) at room temperature. An isocratic chromatographic method was used for affinity separation that consists of: 1) 0-10 min 100% mobile phase A for retention of glycated peptides by interaction between boronate ligands and 1,2-cis diol groups of glucose moieties, with elution of non-glycated peptides; 2) 10-20 min 100% mobile phase B (0.1 M HAc) for elution of glycated peptides; and 3) 20-30 min 100% mobile phase A for the equilibration of the column to the initial conditions. Both the non-glycated and the glycated fractions were collected for subsequent evaporation and reconstitution in 5% ACN/0.1% formic acid. Then, peptides were desalted and preconcentrated prior to LC-MS/MS analysis. This was carried out with C¹⁸ microspin columns (Harvard Apparatus, Holliston, Mass., USA) according to the protocol recommended by the manufacturer, which ends with elution of peptides with 400 μl 50% ACN/0.1% formic acid. This solution was evaporated to dryness for reconstitution with 50 μl 5% ACN/0.1% formic acid.

LC-MS/MS analysis of peptides—Peptides were analysed with a nanoflow HPLC using a Waters NanoAcquity HPLC system (Milford, Mass.) coupled to a hybrid linear ion trap-orbitrap mass spectrometer (Thermo Fisher, San Jose, Calif.) with electrospray ionization in positive mode. The HPLC system included a helium degasser (Michrom SA, Auburn, Calif.). Peptides were trapped on a homemade 100 μm inner diameter 18 mm long precolumn packed with 200 Å (5 μm particle size) Magic C18 particles (C18AO: Michrom) for 12 min. Subsequent peptides separation was on a homemade gravity-pulled 75 μm inner diameter 150 mm long analytical column packed with 100 Å (5 μm particle size) Magic C18 particles (C18AQ: Michrom) and directly interfaced to the mass spectrometer.

For each LC-MS/MS analysis, an estimated amount of 0.5 μg of peptides (0.1 μg/μl) was loaded on the precolumn at 3 ml/min in water/ACN (95/5 v/v) with 0.1% (v/v). After retention, peptides were eluted using an ACN gradient flowing at 220 nl/min with: mobile phase A, water, 0.1% formic acid; mobile phase B, ACN, 0.1% formic acid. The gradient program was as follows: 0 min, A (95%), B (5%); 55 min, A (65%), B (35%); 60 min, A (15%), B (85%); 65 min, A (85%), B (15%); 75-90 min, A (95%), B (5%). The electrospray ionization voltage was applied via a liquid junction using a gold wire inserted into a microtee union (Upchurch Scientific, Oak Harbor, Wash.) located in between the precolumn and analytical column. Ion source conditions were optimized using the tuning and calibration solution recommended by the instrument provider.

Two complementary data-dependent tandem mass spectrometry methods were used for analysis of glycated proteins: MS2 with high-energy collisional dissociation (HCD) as activation mode and MS3 by neutral loss scan with CID as activation mode. In data-dependent HCD-MS2 analysis, fragmentation of the three most abundant precursor ions was carried out on the octopole collision cell attached to the C-trap (normalized collision energy 50 eV) while detection was performed with orbitrap accuracy. The precursor ion isolation window was set to 2 m/z units. MS survey scans were acquired at resolution R=60 000 in profile mode while MS2 spectra were acquired at resolution R=7500. Precursor ions of charge state +2 and higher were included for data-dependent selection. In cases where charge state could not be identified, the most abundant ion was selected for HCD. Data-dependent acquisition was then performed over the entire chromatographic cycle. Data-dependent CID-MS3 neutral loss scan was entirely carried out in the linear trap with three steps: 1) first fragmentation of medium collision energy (35 eV) to promote the cleavage of the glucose moiety (−162.05 Da, that correspond to −81.02 and −54.01 Da for doubly and triply charged peptides, respectively) or an intermediate fragmentation of the glucose molecule (−84.04 Da, that correspond to −42.02 and −28.01 Da for doubly and triply charged peptides, respectively); 2) isolation of the ions in which one of the neutral losses is detected; and (3) fragmentation of the isolated peptide with a medium collision energy (35 eV). Similarly, the precursor ion isolation window was set to 2 m/z units and MS survey scans were acquired at resolution R=60 000 in profile mode. In this case, MS2 and MS3 acquisition was carried out with ion trap resolution. Precursor ions of charge state +2 and higher were included for data-dependent selection. In cases where charge state could not be identified, the most abundant ion was selected for CID. Data-dependent acquisition was then performed over the entire chromatographic cycle.

Data analysis—After data-dependent acquisition, a post-acquisition workflow was initiated specifically for each MS operation mode. For HCD-MS2 experiments, the workflow was based on the detection of precursor ions in an accurate way. This workflow consisted of three major steps. First, peak detection was performed over the entire chromatographic elution profile for each precursor ion scan. This step was performed using the feature-detection software Hardklör (28). During this step, a list of potential monoisotopic precursors for each precursor ion scan was created. Second, tandem mass spectral data were converted into peak lists (.dta files) using the instrument vendor's software (extract_msn.exe; Thermo Fisher). During this step, a .dta file was created for every tandem mass spectrum. This simple text file contains the precursor ion MH⁺ value and charge state (as assumed by the instrument) in the first line, and then a list of fragment ion m/z values and abundance in the remaining lines. If the charge state was not clearly assigned, extract_msn.exe creates one .dta file for a potential +2 charge state ion and one .dta file for a potential +3 charge state ion. In the last step, the measured precursor ion mass and charge given by the instrument (read from the .dta were compared to all possible precursor ions within a given elution time window and precursor ion transmission window. For our system, a peak elution window of ±6 s of the considered tandem mass spectrum and a precursor ion transmission window of ±1.1 m/z units were used. Potential precursor ion peaks detected in more than one MS spectrum were averaged (geometrical mean) if they were observed within a ±5 ppm tolerance. Then, all possible collected precursor ions MH⁺ and charge state values were ranked according to their summed correlation values over the considered time window. In those situations, up to three peaks (the three peaks with highest summed correlation values) were used as potential candidate precursor ions. In the situation where no peak was detected in the considered survey scan windows, the m/z value contained in the original .dta file was kept, with charge states +2 and +3. This last step was performed using a Pen script which is available at the Goodlett laboratory website, http://goodlett.proteomics.washington.edu. For MS3 neutral loss experiments, the same workflow was used except the first step since detection was not carried out with orbitrap accuracy. Therefore, peak lists were created with extract_msn.exe from tandem mass spectral data in the second fragmentation step after neutral loss step.

The resulting dta files for both MS operation modes were searched against UniProt-Swiss-Prot/TrEMBL database (Swiss-Prot Release 56.6 of Dec. 16, 2008, 287 050 entries and TrEMBL release 39.6 of Dec. 16, 2008, 4 988 379 entries) using Phenyx 2.6 (GeneBio, Geneva, Switzerland) operating on a local server. No taxonomy was used for the model protein mixture and Homo sapiens was specified for plasma database searching experiments. Common amino acid modifications for both MS operation modes were carbamidomethylation of cysteines and oxidized methionine, which were set as fixed and variable modifications, respectively. For HCD-MS2 experiments, glycation of lysine and arginine residues or on N-terminal positions (162.052 and 168.072 Da for glycated peptides with [¹²C₆]- or [¹³C₆]-glucose) was selected as variable modification. For MS3 neutral loss experiments, a variable modification as a consequence of glucose fragmentation after neutral loss of 84.04 Da (78.01 Da for K, R and on N-terminal positions) was additionally specified. Endoproteinase Glu-C was selected as enzyme, with three potential missed cleavages as maximum. The peptide and fragment ion tolerance depended on the MS operation mode. For HCD-MS2, peptide and fragment ion tolerance was tuned at 6 ppm. In contrast, these values were 1.1 and 0.8 Da for precursor and fragment ions in MS3 neutral loss. In both modes two sequential search rounds were used. In the first round, two missed cleavages were allowed in normal mode. This round was selected in “turbo” search mode. In the second round, three missed cleavages were allowed in half-cleaved mode. The minimum peptide length allowed was six for both rounds. The acceptance criteria were slightly lowered in the second round search. These were for HCD-MS2 experiments: AC score 9.7, peptide Z-score 9.7, peptide p value 1 10⁻⁷ for round 1; AC score 9.5, peptide Z-score 9.5, peptide p value 1 10⁻⁶ for round 2, corresponding to an estimated false positive ratio of less than 1%. For MS3 in neutral loss experiments, these parameters were changed to AC score 7.0, peptide Z-score 7.0, peptide p value 1 10⁻⁶ for round 1; AC score 6.5, peptide Z-score 6.5, peptide p value 1 10⁻⁵ for round 2, corresponding to an estimated false positive ratio of less than 1%. False positive ratios were estimated using a reverse decoy database. This estimation was performed using separate searches in the reverse database to keep the database size constant. This involved a slight underestimation of the estimated false positive ratio (29). In case of several matching entries, Swiss-Prot entries were preferred to TrEMBL entries. All data were acquired in triplicate (three analytical injections of the same sample) and analysed in an independent manner.

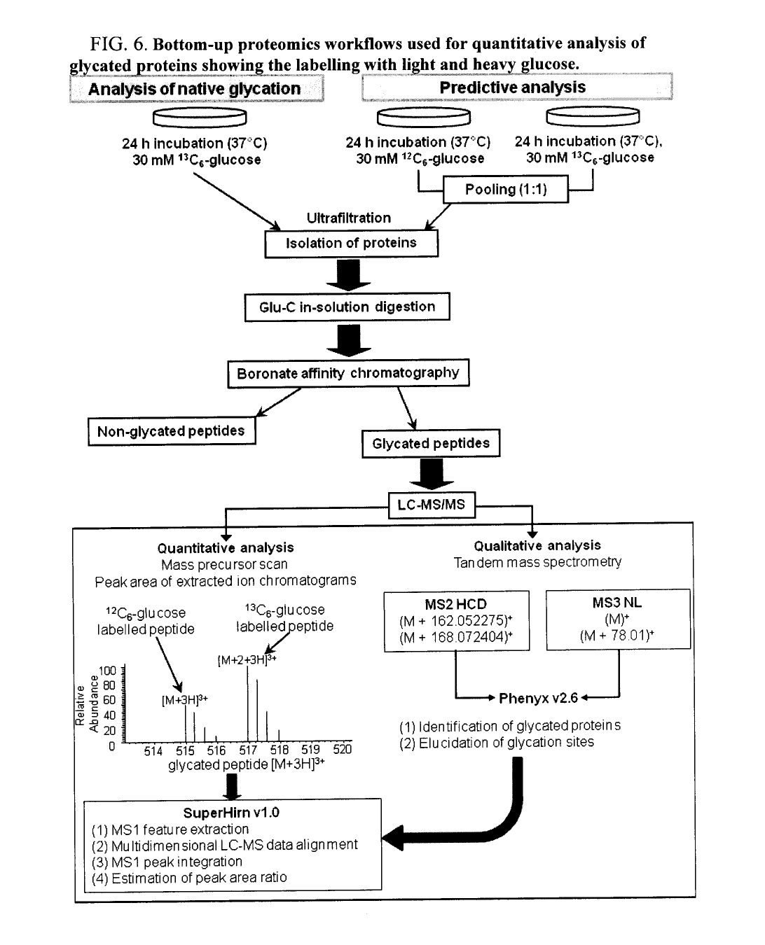

Peptide quantification—Quantitation of glycated proteins was possible as after enzymatic digestion, the resulting glycated peptides (with addition of 162 mass units) provided doublet signals in precursor MS scan (labelling with light and heavy glucose). The mass shift of the doublet signals depended on the peptide charge and the number of glycation sites. Peptide quantification was carried out by calculation of the ratio between peak areas from extracted ion chromatograms corresponding to both isotopic forms of each glycated peptide. Due to the same physicochemical properties of the two isotopic glycated peptides, these were chromatographically co-eluted providing a doublet signal with a mass shift that depends on the peptide charge and the number of glycation sites. The peptide ratios [¹²C₆]-glucose peptide/[¹³C₆]-glucose peptide were obtained from the average values of intra-run triplicates. As shown in FIG. 6, data treatment was automated using the SuperHirn software (version 1.0) (30), which is freely available together with detailed documentation material on http://tools.proteomecenter.org/SuperHirn.php. The .raw data files were converted to mzXML (31) file format in profile mode and SuperHirn performed the feature extraction and alignment of the replicate runs (SuperHirn used standard Orbitrap settings). The post-processing of the feature list was performed in the R statistical programming environment (www.r-project.org). The SuperHirn result files were parsed in order to find all heavy-light pairs (within a mass tolerance of 0.01 Da and retention time tolerance of 20 s) that appeared in at least 2 of the replicates. Then, all accepted identifications from the Phenyx excel export were attributed to a heavy-light pair, if such a pair could be detected (˜80% of the cases). Since the retention times were missing in this export, the scan number of each MS2 spectrum had to be converted into the corresponding retention time using a calibration routine. In summary, quantification was performed in MS precursor scan while identification was based on MS/MS data. Both data treatment steps were carried out in an automated manner by generation of an analysis report.

### Results

### Optimization of the Method for the Analysis of Glycated Proteins

Qualitative Analysis by Tandem Mass Spectrometry—The complete workflow for the analysis of glycated proteins, shown in FIG. 6, was optimized using the multistandard of model recombinant proteins and reference plasma. The first step studied was the enzymatic cleavage (data not shown). For this purpose, the influence of two different enzymes, trypsin (cleaving predominantly at the carboxyl side of Lys and Arg residues) and endoproteinase Glu-C (cleaving predominantly at the carboxyl side of Glu residues), was tested. As glucose attachment is selective for Lys and Arg residues, trypsin digestion pattern was affected increasing the number of missed cleaved sites. A high proportion of half-cleaved peptides was also detected. The influence of glucose attachment was less dramatic for endoproteinase Glu-C as identifications of missed cleaved sites and, particularly, half-cleaved peptides were considerably reduced. As enzyme specificity is maintained with endoproteinase Glu-C, this enzyme was selected for this proteomics workflow.

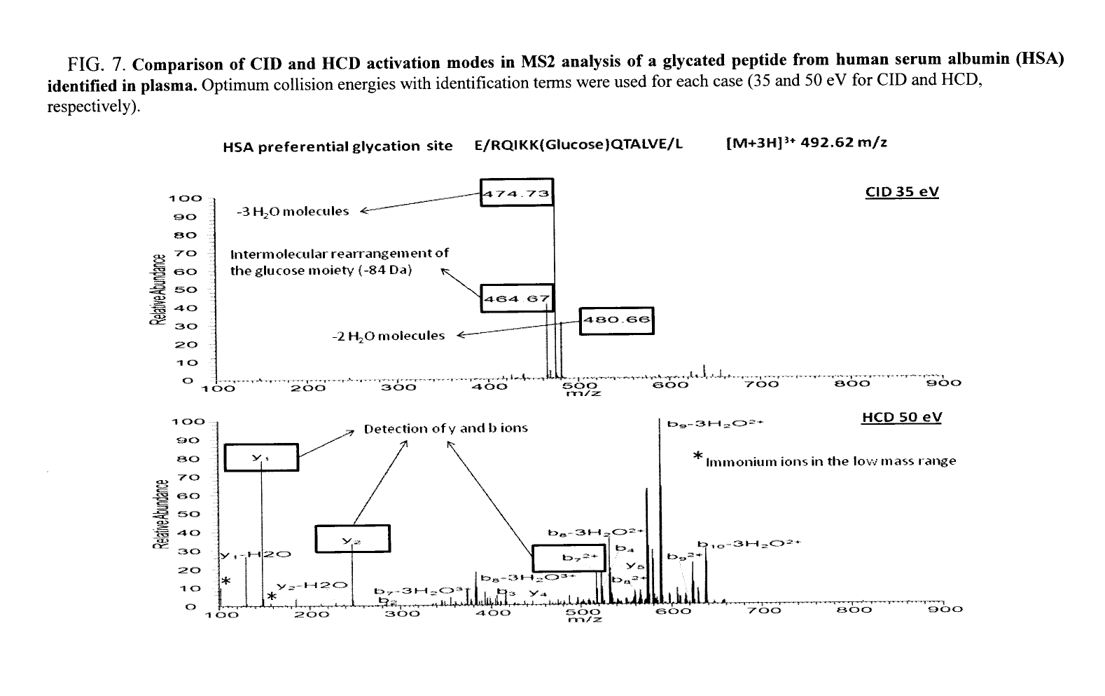

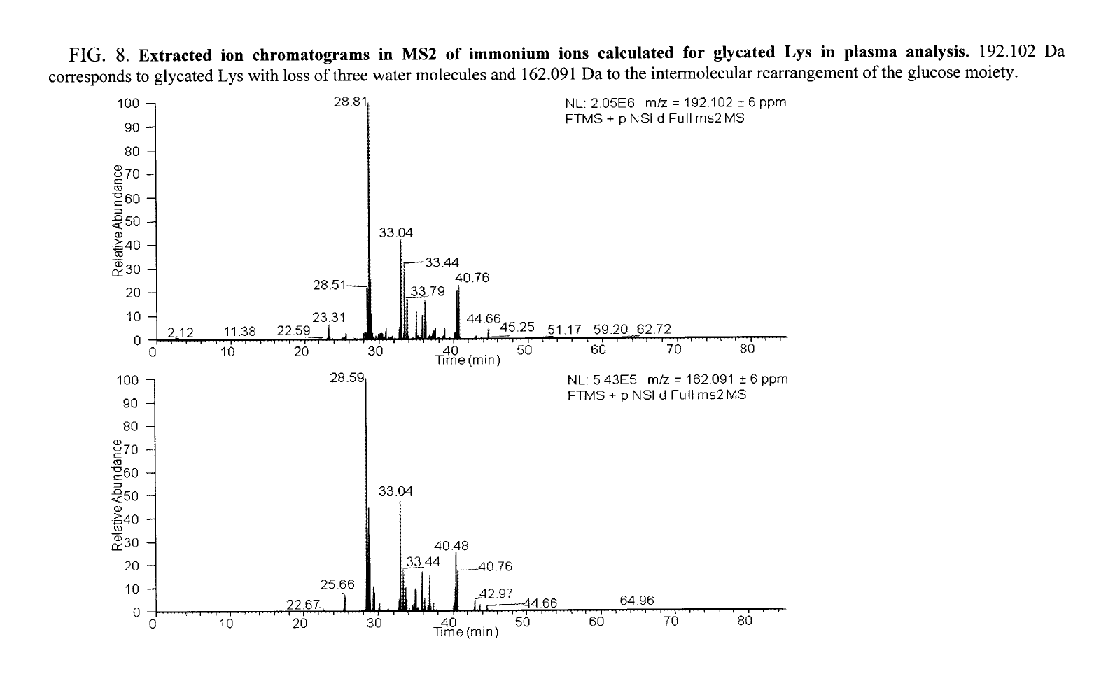

Concerning mass spectrometry, electron transfer dissociation (ETD) (23) and CID in data-dependent MS3 and pseudoMS3 approaches (neutral loss scan and multistage activation, respectively) (24) have proved to be efficient activation modes for identification of glycated peptides. Nevertheless, the use of the orbitrap hybrid mass analyser enables the application of an additional ion dissociation mode, which has not been tested yet for glycation analysis. This is the HCD mode that is characterized by its performance in an additional octopole collision cell attached to the C-trap using nitrogen as collision gas. The use of nitrogen results in a more energetic fragmentation than helium-based dissociation occurring in CID. In addition, HCD is a fast activation mode as compared to CID, which enables to reach high vibrational energies per bond before dissociation of the target molecular ion. As a result, high-quality fingerprinting spectra are obtained which enhances the identification of glycated peptides. FIG. 7 compares CID and HCD generated spectra by activation of two representative glycated peptides corresponding to human serum albumin (HSA) identified in plasma. Optimum collision energies in terms of identification were used for each case (35 and 50 eV for CID and HCD, respectively). HCD spectrum provides a high-quality fingerprinting of the peptide backbone with identification of y and b ions. One other benefit of HCD-MS2 is the detection of immonium ions that can be clearly visualized in the low-mass range to confirm peptide identification. Immonium ions have proved its particular interest to pinpoint the existence of modified amino acids such as phosphorylated Tyr and carboxymethylated Cys (34). By similarity, this can be applied to glycated Lys and Arg but considering the losses detected in glycated entities, the loss of three water molecules and the intermolecular rearrangement of the glucose moiety (−54.031 and −84.042 Da). Thus, immonium ions calculated for glycated Lys were 192.102 and 162.091 Da whereas for Arg were 237.135 and 207.124 Da, respectively. Due to the selectivity of these ions, glycated peptides can be localized by extracting ion chromatograms in MS2 as shown in FIG. 8 for lysine glycated peptides.

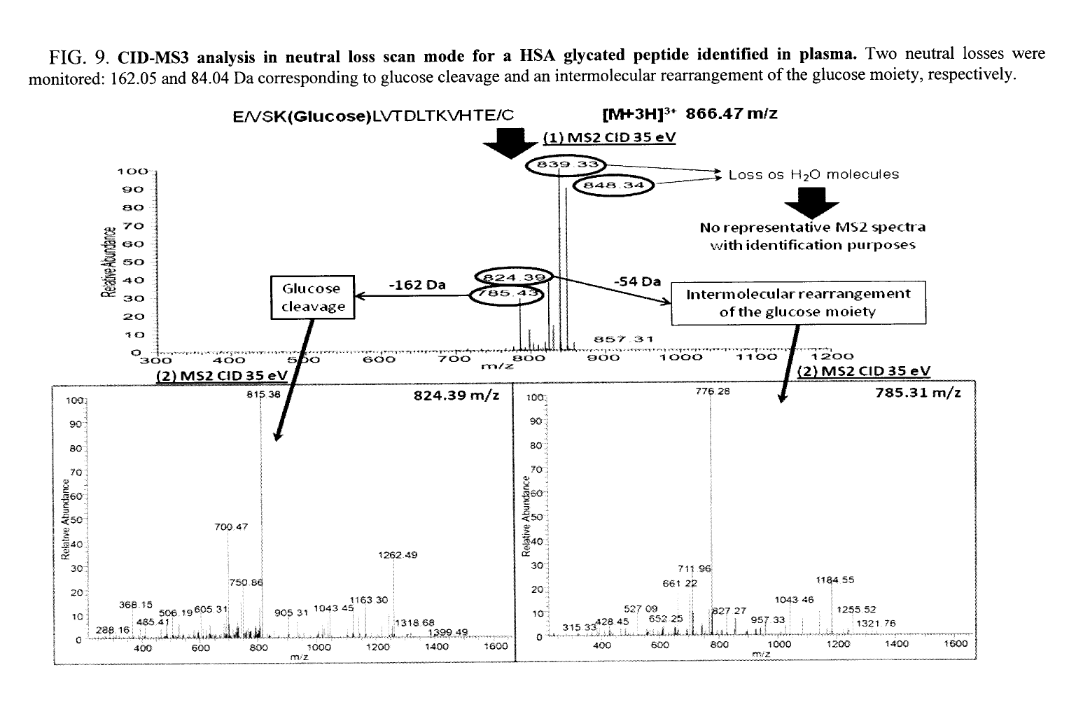

Analysis in MS2 was complemented by MS3 in neutral loss scanning FIG. 9 shows a representative example for a glycated peptide from serum albumin detected in plasma analysis. The precursor ions were activated in a first step by CID (35 eV) to promote the loss of specific neutral fragments. The fragmentation scheme for this peptide illustrates the characteristic neutral losses obtained by the different approaches. These neutral losses fit with the cleavage of the glucose moiety (162.05 Da), dehydration of up to three water molecules (18.01, 36.02 and 54.03 Da) to form pyrylium ion, and dehydration with additional loss of a formaldehyde molecule to generate the furylium and immonium ions (84.04 Da). After this fragmentation, ions formed by loss of 162.05 and 84.04 Da are isolated in the ion trap for a second fragmentation, which now generates representative fingerprinting spectra with identification purposes as shown in FIG. 9. Ions formed by the other neutral losses (18.01, 36.02 and 54.03 Da) are excluded, as they do not provide MS3 spectra useful for identification. Since these ions still contain labile parts in their structure, the MS3 spectra generated are similar to CID-MS2 spectra of glycated peptides. Neutral loss analysis was carried out in the ion trap to avoid transfers of ions to the orbitrap analyser with the subsequent decrease of sensitivity.

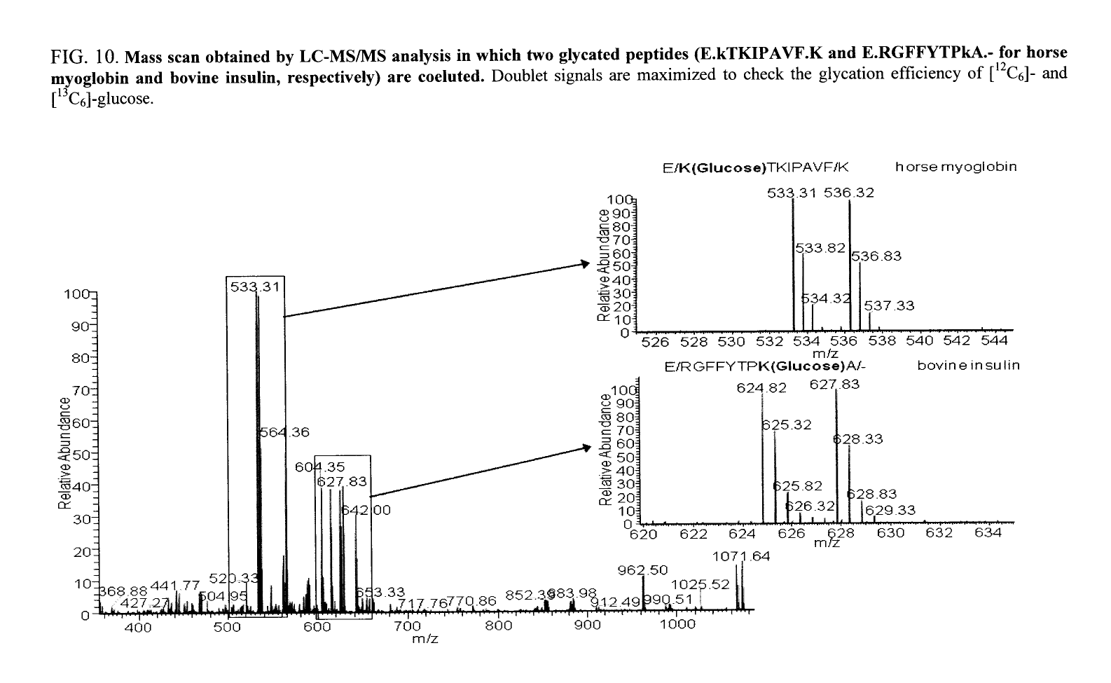

Quantitative analysis based on the GIL approach—As shown in FIG. 6 quantitation is based on the differential labelling with isotopic sugars under physiological conditions to compare between biological states. As it was previously emphasized labelling with both isotopic glucose molecules enables the detection of glycated peptides by mass spectrometry as they provide a doublet signal in MS scan (+6 Da per glycation site). The quantitative approach was initially optimized with the multistandard of model recombinant proteins, which was analysed with the protocol exposed in FIG. 6. FIG. 10 shows one of the MS scans obtained a 26.57 min retention time by RPLC in which two doubly charged glycated peptides were co-eluted.

The doublet signals are 533.31/536.32 m/z and 624.82/627.83 m/z, with a mass shift of 3 Da, which is indicative of doubly charged glycated peptides. The peptide at 533.31 m/z corresponded to a horse myoglobin glycated peptide while that at 624.82 m/z was identified as a bovine insulin glycated peptide. This experiment was obtained by incubation of the standard composed of four reference proteins with “light” and “heavy” glucose and subsequent pooling with a 1:1 ratio. The intensity of MS signals corresponding to the two versions of the peptide labelled with both isotopic glucose forms was practically the same. Particularly, the ratios between peak areas were 0.965±0.010 and 1.018±0.025 for myoglobin and insulin glycated peptides, respectively. These values were obtained by analysis of three technical replicates.

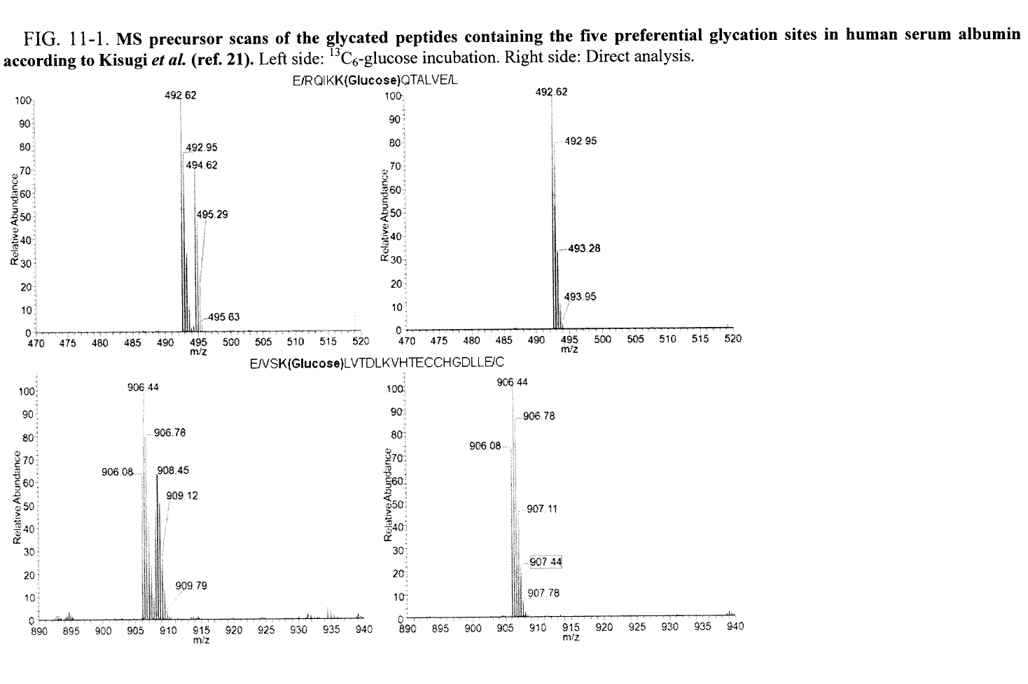

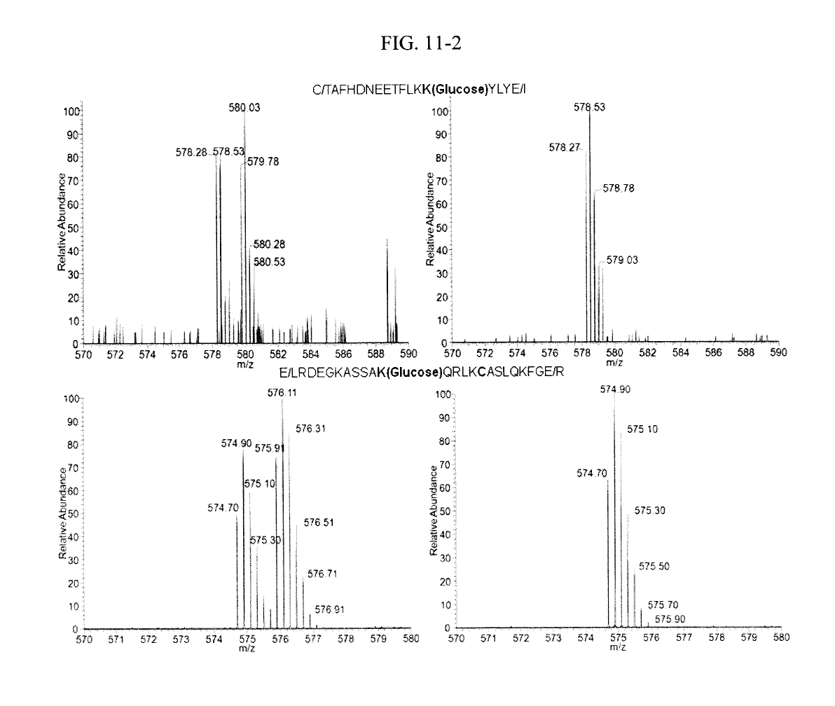

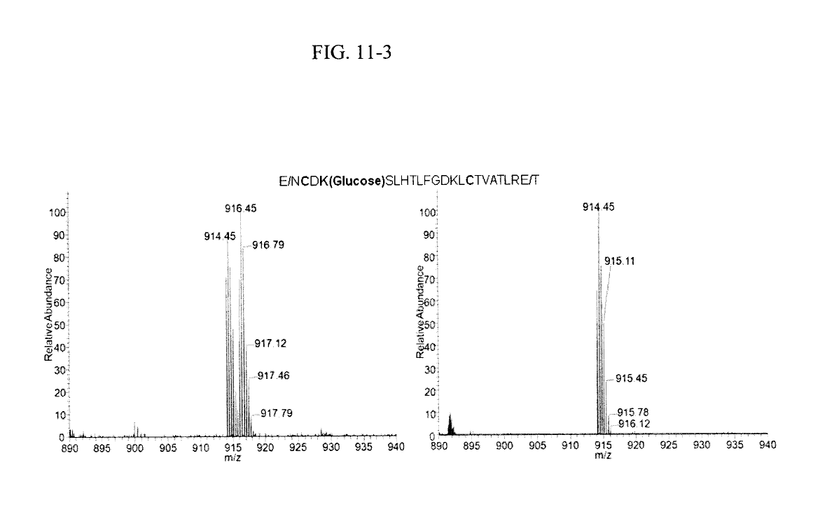

Tests of the optimized protocol to human plasma—After optimization of the glucose labelling principle, the next step was to test it with a relatively complex biofluid as human plasma. For this purpose, two aliquots of plasma (50 μl each) were independently incubated with 30 mM [¹²C₆]-glucose and [¹³C₆]-glucose for 24 h at 37° C. In this case, each aliquot was analysed separately using the workflow exposed in FIG. 6. After incubation and ultrafiltration, an aliquot of 2-mg total protein content quantified with the Bradford assay was taken for enzymatic digestion to continue with the analytical workflow. The aim for this experiment was to validate the applicability of doublet signal detection as an analytical tool for the assessment of glycation. FIG. 11 shows the MS precursor scans of five glycated peptides that contain the preferential glycation sites of human serum albumin according to the literature. These glycation sites have been found at concentrations within the range 8-0.8% in healthy patients according to Kisugi et al. who found a total concentration of glycated albumin of 14.7% as compared to diabetic patients with a total content of glycated albumin around 25.4% (21). These five preferential glycation sites were detected in the aliquot incubated with [¹²C₆]-glucose. The intensity of these signals is the contribution of the native glycated protein existing in plasma and that as a consequence of the glucose stimuli (30 mM incubation for 24 h at 37° C.).

Concerning the experiment based on incubation with [¹³C₆]-glucose, the same peptides provided doublet signals that favour their identification. In this case, the signals corresponding to peptides labelled with “heavy” sugar are caused by glucose perturbation mimicked with in vitro incubation. On the other hand, the signals provided by glycated peptides with “light” glucose are indicative of the native concentration of them in plasma.

This experiment enables to validate the principle of isotopic sugar labelling as a possibility for quantitation of glycated proteins and points out two significant applications of this quantitative approach that are subsequently exposed.

### Detection, Quantitation and Prediction of Human Glycated Plasma Proteins

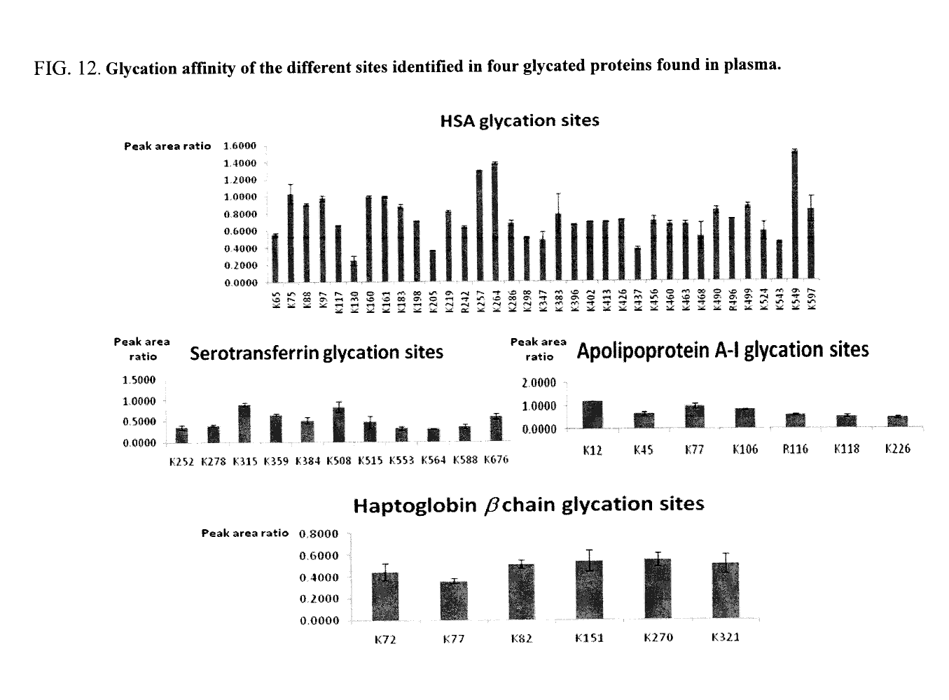

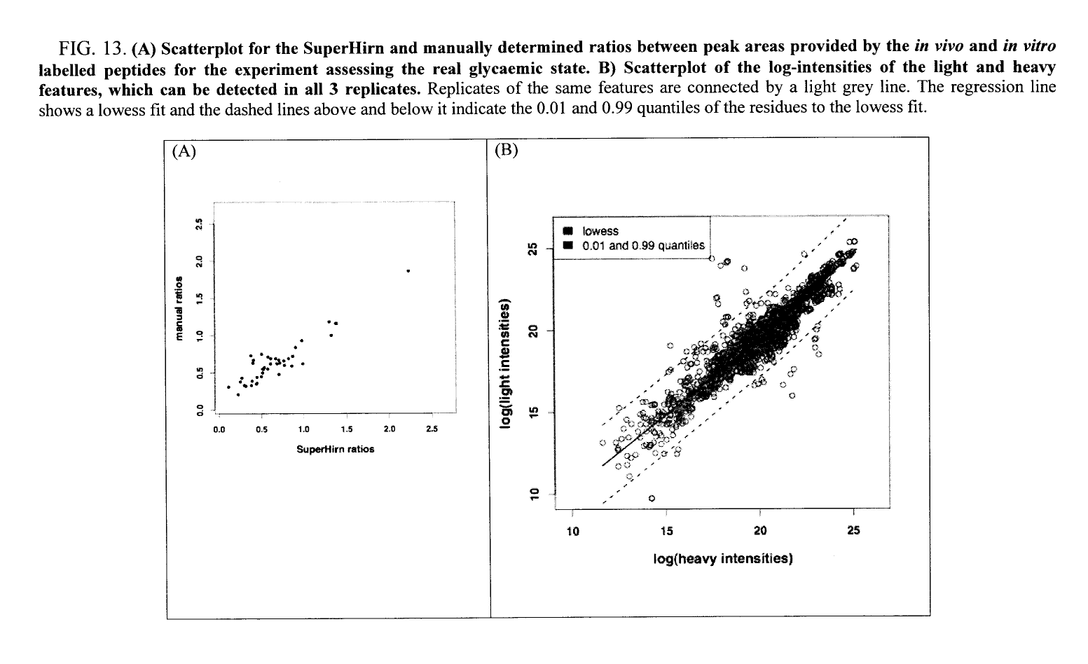

Assessment of the native level of plasma protein glycation—The application of the optimized protocol to plasma enables to obtain a global view about the glycaemic state of a potential patient. This analysis provides the profile of glycated proteins identified together with information about glycation sites as shown in Table 2 for the reference plasma used in this research. A total of 35 proteins was found to be glycated in the reference plasma sample without any pre-fractionation step at the protein level. The proposed methodology is able to detect 113 different glycation sites, which is of particular interest as each glycation site could have a different impact on the biological function of proteins. For instance, 35 different glycation sites were identified for HSA. As it was previously indicated, previous studies have identified preferential glycation sites for HSA in Lys residues located in positions 549, 257, 264, 468 and 160. This approach enables to compare the efficiency of the sugar attachment on the different glycation sites. For this purpose, values of the ratio between the peak areas of the in vivo and in vitro glycated peptides (labelled with [¹²C₆]- and [¹³C₆]-glucose) are estimated using extracted ion chromatograms. FIG. 12 compares the glycation efficiency for the different sites detected in four representative plasmatic proteins as a function of areas ratio. The resulting graphs provide structural information about localization of preferential glycation sites that is of great interest to elucidate the biological effect on the protein function. It can be deduced from these representations the affinity glycation sites for HSA (Lys 549, 264, 257, 75, 160, 161 and 97 as the preferential glycation sites) as well as for other plasma proteins such as Serotransferrin (Lys 315 and 508), Haptoglobin (Lys 270 and 151) or Apolipoprotein A-I (Lys 12 and 77). Table 2 additionally includes quantitative information for each of the glycated peptides identified in plasma (in relative terms as the ratio between peak areas provided by [¹²C₆]- and [¹³C₆]-glucose labelled peptides). These ratios were automatically calculated using SuperHirn. In order to evaluate the automated analysis with SuperHirn for the experiment described above we plotted the ratios obtained by manual integration against those calculated from the SuperHirn result files (FIG. 13A) revealing a high correlation between the two values (Pearson correlation=0.91). FIG. 13B plots the glucose labelled ones. The features with deviating [¹²C₆]-glucose/[¹³C₆]-glucose ratios are clearly pointed out from the cloud of background ratios. The width of the cloud indicates the deviation in log intensities even if no real change is present. The points belonging to the replicates of the same feature are connected by a grey line, which shows that replicates are very close and therefore that the analytical method possesses a good technical precision. The deviation between replicates is much smaller than the ‘biological’ deviation between different features.

Prediction of the glycation site state as response to glucose stimuli—In this study, glucose perturbations were assessed by independent incubation of two plasma aliquots with [¹²C₆]- and [¹³C₆]-glucose. A glucose concentration of 30 mM was selected for incubation mimicking a glucotoxicity perturbation. After incubation, both aliquots were pooled at 1:1 ratio for standardization prior to proteomics analysis following the reported protocol. As shown in Table 3, 50 glycated proteins were identified with this analysis. As compared to the analysis based on exclusive incubation with [¹³C₆]-glucose, 20 new glycated proteins were identified. Additionally, a total number of 161 glycation sites were detected. Most of these identifications corresponded to singly glycated peptides. Nevertheless, it is worth emphasizing the detection of peptides containing two different glycation sites, which were undetectable in the analysis of native glycation. For this reason, they could be considered as potential biomarkers to assess glucotoxicity levels in clinical patients. Concerning data treatment, the signals corresponding to peptides labelled with [¹³C₆]-glucose are representative of the 30 mM glucose stimuli. On the other hand, the signals provided by peptides labelled with [¹²C₆]-glucose are contribution of two different sources: native glycated proteins present before incubation (equal contribution from both aliquots) and those generated as a consequence of the [¹²C₆]-glucose stimuli for 24 h. Therefore, this approach enables to differentiate glycated proteins formed as a result of the glucotoxic perturbation in relative terms. For doubly glycated peptides, we can discriminate between: those in vitro labelled with [¹²C₆]- or [¹³C₆]-glucose as a result of the stimuli and, those that were singly labelled with [¹²C₆]-glucose before the stimuli and are secondly labelled due to the stimuli with [¹²C₆]- or [¹³C₆]-glucose.

This prediction approach enables the assessment of the impact of glycaemic disturbances for the different glycation sites. Table 3 also evaluates the effect of the 30 mM glucose stimuli for each glycated peptide (right column) by comparison with the native glycation as reference. This parameter was calculated with the following expression:

$$glucotoxic effect ( \% ) = \frac{Peak {area}^{heavy glycated peptide}}{( Peak {area}^{light glycated peptide} - Peak {area}^{heavy glycated peptide} ) / 2} \cdot 100$$

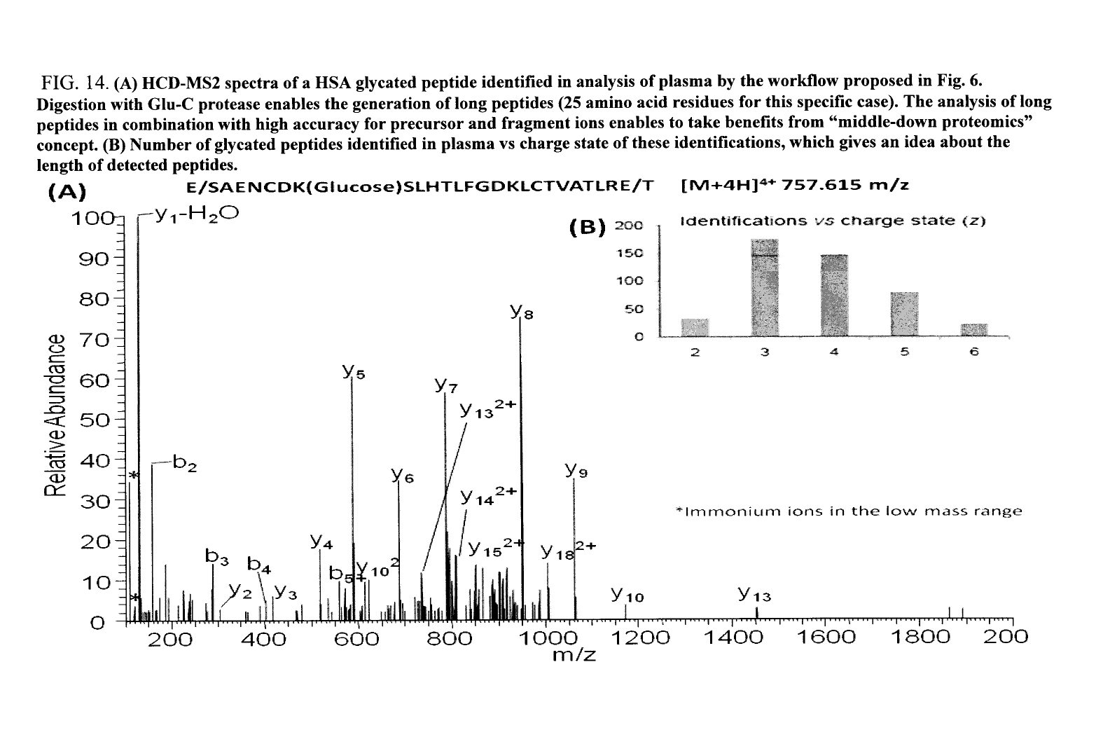

As an example, preferential glycation sites in HSA such as Lys549, Lys264 and Lys257 experienced glucotoxic effect between 36.2 and 56.8% in plasma subjected to 30 Mm glucose exposition for 24 h. FIG. 14 shows doublet signals in MS precursor scan corresponding to different glycated peptides identified in plasma by application of the predictive approach. On the other hand, a higher impact is observed in potential sites with lower glycation affinity such as Lys524 and Lys543, which showed glucotoxic effects of 229.5 and 316.2%, respectively. Additionally, the predictive approach enables the identification of potential glycation targets such as the glycated peptides containing Arg242 in HSA, Arg273 in serotransferrin or Lys37 in Ig κ chain C region. As can be seen, peak area ratios of the [¹²C₆]- and [¹³C₆]-glucose labelled peptides were close to one, which is indicative of a labelling only during the glucotoxic perturbation. As a similar labelling efficiency has been observed for [¹²C₆]- and [¹³C₆]-glucose, the result for these peptides proves that this is a new potential target for glycation under these specific conditions (30 mM glucose exposition for 24 h).

### Discussion

### Optimization of the Method for Analysis of Glycated Proteins

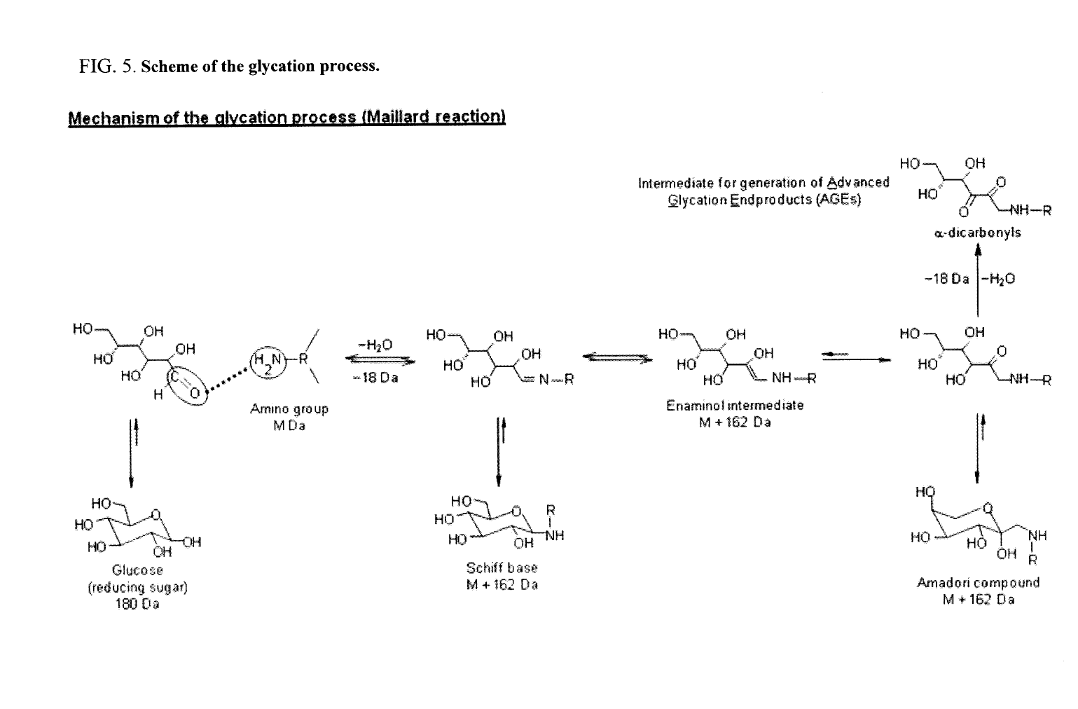

This research describes the development of an application for qualitative and quantitative analysis of glycated proteins in human plasma. There are several reasons that have contributed to the lack of methods for identification and quantitation of glycated proteins. Among them, we have to emphasize the modification of enzymatic digestion patterns and the lack of strategies to detect glycated proteins present in humans at low concentrations. Due to the influence of glycation on trypsin enzymatic digestion, the implementation of an alternative protease such as Glu-C has proved to be an effective way to avoid pattern modifications. In this way, enzymatic specificity can be maintained for identification of glycated peptides by minimizing the generation of missed cleavage sites and half-cleaved peptides. The development of selective and sensitive strategies for the detection of glycated proteins has been accomplished by the advances experimented by mass spectrometry in the last years. Electron transfer dissociation has proved to be an efficient activation mode for identification of glycated peptides by tandem mass spectrometry. Nevertheless, ETD instrumentation is less distributed and frequently characterized by a significant decrease of sensitivity as compared to CID, which was the initial activation mode for analysis of glycated peptides. However, CID-based fragmentation tends to dissociate Amadori compounds (see FIG. 5), which results in low-quality peptide fingerprinting due to a poor production of sequence specific ions from the peptide backbone. Signals corresponding to ions generated by losses of specific neutral fragments dominate preferentially the mass spectrum with a reduced success in peptide identification (35, 36). Zhang et al. have recently used this well-characterized knowledge in data-dependent MS3 by neutral loss scan and pseudo-MS3 by multistage activation (24). Both advanced approaches take benefit from a first ion dissociation step that promotes labile neutral losses in order to increase the MS/MS quality of spectra provided by a second dissociation step. For this reason, data-dependent MS3 seems to be especially interesting in the characterization of PTMs and an efficient alternative to ETD to increase identification coverage in glycation analysis.

In the present study, a combination of a MS2 mode with HCD activation and CID-MS3 by neutral loss scan is proposed for qualitative analysis of glycated proteins. The high accuracy in HCD-MS2 mode for precursor and fragment ions is crucial to achieve a high identification level (37, 38) for characterization of glycation, particularly, if Glu-C is used for hydrolysis. This enzyme enables to generate long peptides such as the glycated peptide shown in FIG. 14A that was identified in the plasma analysis. In this way, the analysis of long peptides (25 amino acid residues for this specific case) with high accuracy enables to increase sequence coverage resulting in high score values. This approach corresponds to the concept of middle-down proteomics defined by Mann et al. as an alternative to take benefits from precision in proteomics (39, 40). Besides, FIG. 14B correlates the number of glycated peptides identified in plasma with its length through the charge state of these identifications. As can be seen, most of the peptides were identified with a charge state above +3 with a significant number of identifications for charge states +4 and +5.

The CID-MS3 mode is a complementary approach to HCD-MS2 as the former is particularly useful for identification of glycated peptides with charge states (+2) and (+3). As an example to evaluate this complementary application, both MS modes were compared in terms of identification of glycation sites. Thus, if a total of 113 different glycation sites were identified in the analysis of plasma, 64% of them were detected with HCD-MS2 and 46.9% with neutral loss scan. These results justify the complementary application of both MS modes in order to increase the identification capability.

The optimization of the overall method was completed by tests to validate the quantitative approach based on glycation isotopic labelling using [¹³C₆]-glucose. These tests were carried out with a standard of recombinant proteins to ensure the absence of glycation. The provided results proved that both isotopic glucose forms possess similar glycation efficiency, which is derived from the peak areas of the extracted chromatograms corresponding to the precursor ions of the [¹²C₆]- and [¹³C₆]-glucose labelled peptides. Evidently, this is a critical aspect for the implementation of isotopic glucose labelling as a quantitative approach.

### Applicability of the Quantitative Approach

Application to the human plasma glycated proteome—As it was previously indicated, any protein can be glycated. However, the reference method for the assessment of the glycaemic control of a patient is the measurement of HbA1c concentration. In addition to be exclusively focused on one protein, the erythrocyte lifespan (˜120 days) defines HbA1c as a long-term indicator of the patient state (41-43). It is clearly evident that the overall profiles of glycated proteins represent a more complete indicator of the glycaemic state of a particular patient. This information can be achieved with the approach based on incubation with [¹³C₆]-glucose as this provides indirectly a view about the current glycaemic state of a potential patient. As [¹²C₆]-glucose concentration is not modified a profile of glycated proteins that are present in a target sample is obtained.

The ratio between peak areas corresponding to the peptides labelled with [¹²C₆]- and [¹³C₆]-glucose provides additional quantitative information in relative terms. Peptides labelled with “heavy” glucose are considered as internal standards with the particularity that these isotopic forms are generated mimicking physiological conditions. Therefore, in vitro labelling with [¹³C₆]-glucose depends on the sample properties such as proteins content or pathological factors affecting glycation. The application of this approach is useful to estimate relatively the extent of glycation for each potential attachment site. In addition, the isotopic glucose labelling is valid as a quantitative approach to compare between two glycation states for the same or different patients.

Prediction of the glycaemic state as response to glucose stimuli—The mechanism of the glycation process (see FIG. 5) has clearly exposed the selectivity of the reaction. In general terms, amino groups with lower pKₐ values should be expected to be more reactive towards glycation because of their greater nucleophilicity. However, there are additional factors that point at the Amadori rearrangement as the critical step to set the site specificity (44). Thus, the properties of nearby amino acids seem to play a relevant role in the potential attachment of sugars to Lys residues. For instance, positively charged amino acids located close to a Lys residue have been proposed to exert a catalytic action for glycation (45). Also, the presence of a His residue close to a Lys promotes its glycation in primary or 3D structures (44, 46). On the other hand, Baynes et al. reported a partial inhibitory effect of Lys glycation due to formation of hydrogen bonds with other amino acids (47). Recently, Johansen et al. have developed a sequence-based predictor of glycation by investigation of ε amino groups of lysines (48). As a result of the statistical analysis, acidic amino acids, mainly Glu and Lys residues, were found to catalyze the glycation of nearby Lys. The catalytic acidic amino acids were found mainly C-terminally from the glycation site, whereas the basic Lys residues were mainly N-terminally found. This in-silico predictor, which is available at www.cbs.dtu.dk/services/NetGlycate-1.0, is the only tool for analysis of non-enzymatic glycation of proteins with predictive purposes. The only limitation is that it is restricted to lysine glycation and, therefore, it does not take into account glycation in arginine residues or in N-terminal position.

The predictive approach here proposed is based on the differential labelling with [¹²C₆]-glucose and [¹³C₆]-glucose and considers all glycation possibilities. As glucose labelling is performed by incubation under physiological conditions, glycation of proteins is mimicked in natural terms. As it has been proved, this fact can be employed for the evaluation of the impact of glucose concentrations on identified sites. This information is collected in Table 3 for each identified glycation site, which was obtained by comparison to native conditions. This approach also enables the identification of new glycation targets for a certain glucotoxic incidence, which is of valuable interest for search of biomarkers by application to a specific pathological disorder.

It can be concluded that an approach for qualitative and quantitative analysis of glycated proteins has been here developed to characterize this undesired PTM. Qualitative analysis, by HCD-MS2 and CID-MS3 operational modes, enabled the identification of glycated proteins in plasma as well as the elucidation of glycation sites. The latter is crucial in order to know the effect of the sugar attachment on the biological function of the protein. Quantitative analysis was accomplished by partial labelling of proteins with ¹³C₆-glucose to discriminate from native glycated proteins labelled with ¹²C₆-glucose. Labelling was performed by physiological incubation taking into account the chemoselective character of glycation. The resulting method was tested by analysis of native glycated proteins in plasma as well as predictive analysis of glycation sites under high glucose concentrations, which is of great interest in clinical applications.

TABLE 2
Glycated proteins identified in plasma analysis with information about the glycation
sites and m/z value of the precursor ion corresponding to the glycated peptides.
Quantitative data are based on peak area ratio between ¹²C₆- and ¹³C₆-glucose labelled
peptides with standard deviation estimated by measurement of three analytical replicates.
The last two columns indicate the MS-mode that detected each peptide.

| | | | | m/z | Peak area | | | |
|---|---|---|---|---|---|---|---|---|
| protein | peptide | gly site | charge | 12Glu | ratio | SD | HCD | NL |
| Serum albumin | I/AFAQYLQQCPEEDHVKLVNE/V | K65 | 3 | 867.08 | 0.5569 | 0.0136 | | X |
| | E/VTEFAKTCVADESAE/N | K75 | 2 | 909.9 | 1.0333 | 0.1127 | | X |
| | E/NCDKSLHTLFGDKLCTVATLRE/T | K88 | 3 | 914.45 | 0.9077 | 0.0149 | X | X |
| | E/SAENCDKSLHTLFGDKLCTVATLRE/T | K97 | 3 | 1009.82 | 0.9810 | 0.0330 | X | X |
| | E/TYGEMADCCAKQEPERNE/C | K117 | 3 | 783.98 | 0.6590 | 0.0076 | X | |
| | E/RNECFLQHKDDNPNLPRLVRPE/V | K130 | 5 | 582.69 | 0.2505 | 0.0553 | X | |
| | C/TAFHDNEETFLKKYLYE/I | K160 | 4 | 578.28 | 0.9956 | 0.0142 | X | X |
| | E/VDVMCTAFHDNEETFLKKYLYE/I | K161 | 3 | 972.45 | 0.9949 | 0.0101 | | X |
| | E/LLFFAKRYKAAFTECCQAADKAACLLPKLDE/L | K183 | 2 | 934 | 0.8818 | 0.0236 | X | |
| | E/CCQAADKAACLLPKLDE/L | K198 | 3 | 708.99 | 0.7086 | 0.0032 | X | |
| | E/CCQAADKAACLLPKLDELRDE/G | K205 | 3 | 880.08 | 0.3667 | 0.0060 | X | |
| | E/LRDEGKASSAKQRLKCASLQKFGE/R | K219 | 5 | 574.7 | 0.8248 | 0.0110 | X | X |
| | E/RAFKAWAVARLSQRFPKAE/F | R242 | 3 | 798.77 | 0.1415 | 0.0152 | X | X |
| | E/VSKLVTDLTKVHTE/C | K257 | 3 | 577.98 | 1.2937 | 0.0124 | X | X |
| | E/VSKLVTDLTKVHTECCHGDLLE/C | K264 | 3 | 906.08 | 1.3840 | 0.0126 | X | X |
| | E/CADDRADLAKYICE/N | K286 | 2 | 931.4 | 0.6850 | 0.0282 | X | |
| | E/NQDSISSKLKE/C | K298 | 6 | 705.85 | 0.5170 | 0.0087 | | X |
| | E/AKDVFLGMFLYE/Y | K347 | 2 | 805.89 | 0.4879 | 0.0933 | | X |
| | E/KCCAAADPHECYAKVFDE/F | K383 | 3 | 778.32 | 0.7851 | 0.2300 | X | X |
| | D/PHECYAKVFDE/F | K396 | 3 | 778.65 | 0.6628 | 0.0057 | | X |
| | E/FKPLVEEPQNLIKQNCE/L | K402 | 3 | 750.04 | 0.6924 | 0.0045 | X | X |
| | E/FKPLVEEPQNLIKQNCE/L | K413 | 3 | 750.04 | 0.6924 | 0.0045 | X | |
| | E/LFEQLGEYKFQ/N | K426 | 2 | 782.38 | 0.7172 | 0.0036 | | X |
| | Q/NALLVRYTKKVPQVSTPTLVE/V | K437 | 2 | 1259.71 | 0.3781 | 0.0199 | X | X |
| | E/VSRNLGKVGSKCCKHPE/A | K456 | 4 | 530.26 | 0.7064 | 0.0476 | X | |
| | E/VSRNLGKVGSKCCKHPE/A | K460 | 4 | 530.26 | 0.6713 | 0.0275 | X | X |
| | E/VSRNLGKVGSKCCKHPE/A | K463 | 4 | 530.26 | 0.6713 | 0.0275 | X | X |
| | H/PEAKRMPCAEDYLSVVLNQLCVLHE/K | K468 | 3 | 1050.94 | 0.5233 | 0.1586 | X | |
| | E/KTPVSDRVTKCCTE/S | K490 | 3 | 614.96 | 0.8258 | 0.0377 | | X |
| | E/KTPVSDRVTKCCTE/S | K496 | 3 | 614.96 | 0.7236 | 0.0064 | X | |
| | E/KTPVSDRVTKCCTE/S | K499 | 3 | 614.96 | 0.8734 | 0.0311 | X | |
| | E/SLVNRRPCFSALEVDETYVPKEFNAE/T | K524 | 3 | 1078.19 | 0.5799 | 0.1059 | X | |
| | E/TFTFHADICTLSEKE/R | K543 | 3 | 654.3 | 0.4466 | 0.0101 | X | X |
| | E/RQIKKQTALVE/L | K549 | 3 | 492.62 | 1.4990 | 0.0226 | | X |
| | E/GKKLVAASQAALGL/- | K597 | 2 | 745.44 | 0.8320 | 0.1510 | | X |
| Serotransferrin | E/LLCLDNTRKPVDE/Y | K252 | 2 | 846.43 | 0.3625 | 0.0484 | | X |
| | E/YKDCHLAQVPSHTVVARSMGGKEDLIWE/L | K278 | 3 | 1130.21 | 0.3978 | 0.0208 | X | |
| | E/FQLFSSPHGKDLLFK/D | K315 | 3 | 643.01 | 0.9035 | 0.0459 | | X |
| | E/CKPVKWCALSHHE/R | K359 | 4 | 454.21 | 0.6489 | 0.0294 | X | |
| | E/RLKCDEWSVNSVGKIE/C | K384 | 3 | 814.03 | 0.5152 | 0.0767 | X | |
| | E/FFSEGCAPGSKKDSSLCKLC/M | K508 | 3 | 814.37 | 0.8316 | 0.1272 | X | |
| | E/GCAPGSKKDSSLCKLCMGSGLNLCEPNNKE/G | K515 | 4 | 869.14 | 0.4677 | 0.1429 | X | |
| | E/KGDVAFVKHQTVPQNTGGKN/P | K553 | 4 | 572.55 | 0.3167 | 0.0400 | X | |
| | E/KGDVAFVKHQTVPQNTGGKNPDPWAKN/L | K564 | 4 | 774.64 | 0.2980 | 0.0166 | X | |
| | E/LLCLDGTRKPVEE/Y | K588 | 2 | 846.43 | 0.3625 | 0.0484 | | X |
| | E/YVKAVGNLRKCSTSSLLE/A | K676 | | 1094.57 | 0.5894 | 0.0650 | | X |
| Apolipoprotein A-I | /DEPPQSPWDRVKD/L | K12 | 2 | 866.4 | 1.2085 | 0.0143 | | X |
| | E/GSALGKQLNLKLLDNWDSVTSTFSKLRE/Q | K45 | 4 | 821.44 | 0.6681 | 0.0626 | X | |
| | E/QLGPVTQEFWDNLEKE/T | K77 | 2 | 1048.49 | 1.0091 | 0.1084 | | X |
| | E/VKAKVQPYLDDFQKKWQEE/M | K106 | 4 | 636.08 | 0.8426 | 0.0171 | X | |
| | E/MELYRQKVEPLRAE/L | R116 | 4 | 481.75 | 0.6045 | 0.0453 | X | |
| | E/LYRQKVEPLRAELQE/G | K118 | 2 | 1018.05 | 0.5479 | 0.0683 | | X |
| | E/SFKVSFLSALEE/Y | K226 | 2 | 759.88 | 0.4792 | 0.0703 | | X |
| Haptoglobin β chain | K/KQWINKAVGDKLPECE/A | K72 | 2 | 1039.52 | 0.4452 | 0.0774 | | X |
| | K/KQWINKAVGDKLPE/C | K77 | 4 | 447.74 | 0.3599 | 0.0226 | X | |
| | E/KQWINKAVGDKLPECE/A | K82 | 3 | 693.01 | 0.5140 | 0.0350 | X | |
| | E/AVCGKPKNPANPVQR/I | K151 | 4 | 450.24 | 0.5414 | 0.0986 | X | |
| | E/RVMPICLPSKDYAE/V | K270 | 2 | 920.94 | 0.5499 | 0.0602 | | X |
| | E/GSTVPEKKTPKSPVGVQPILNE/H | K321 | 2 | 1234.67 | 0.5113 | 0.0881 | | X |
| α-1-antitrypsin | E/NELTHDIITKFLEN/E | K298 | 2 | 868.45 | 0.7833 | 0.1376 | | X |
| | E/EAPLKLSKAVHKAVLTIDEKGTE/A | K352 | 3 | 880.49 | 0.6272 | 0.1355 | X | |
| | E/EAPLKLSKAVHKAVLTIDEKGTE/A | K355 | 3 | 880.49 | 0.3997 | 0.0495 | X | |
| | E/QNTKSPLFMGKVVNPTQK/- | K404 | 3 | 727.05 | 0.4994 | 0.0506 | X | |
| | N/TKSPLFMGKVVNPTQK/- | K411 | 3 | 646.35 | 0.5308 | 0.0721 | X | |
| Apolipoprotein A-II | E/AKSYFEKSKEQLTPLIKKAGTE/L | K44 | 3 | 886.81 | 0.4970 | 0.0134 | X | |
| | E/AKSYFEKSKEQLTPLIKKAGTE/L | K46 | 3 | 886.81 | 0.3843 | 0.0748 | X | |
| | E/KSKEQLTPLIKKAGTE/L | K54 | 3 | 645.03 | 0.5126 | 0.0315 | X | |
| Haptoglobin-related protein | KQWINKAVGDKLPECE | K77 | 3 | 693.01 | 0.5008 | 0.0059 | X | |
| | KQWINKAVGDKLPECE | K82 | 3 | 693.01 | 0.5008 | 0.0059 | X | |
| | AVCGKPKNPANPVQR | K151 | 4 | 450.24 | 0.5414 | 0.0986 | X | |
| Ig γ-1 chain C region | E/LLGGPSVFLFPPKPKDTLMISRTPE/V | K129 | 3 | 968.19 | 0.5690 | 0.0102 | X | |
| | E/YKCKVSNKALPAPIEKTISKAKGQPREPQVYTLPPSRDE/L | K221 | 6 | 765.25 | 0.3638 | 0.0409 | X | |
| β-2-glycoprotein 1 | ./GRTCPKPDDLPFSTVVPLKTFYEPGEE/I | K19 | 3 | 704.33 | 0.3302 | 0.0683 | X | |
| | E/HSSLAFWKTDASDVKPC/- | K317 | 4 | 811.15 | 0.4049 | 0.0647 | X | |
| Complement factor H | E/VVKCLPVTAPENGKIVSSAMEPDRE/Y | K138 | 4 | 723.11 | 0.5098 | 0.0870 | X | |
| | E/MHCSDDGFWSKEKPKCVE/I | K182 | 4 | 601.26 | 0.4092 | 0.1363 | X | |
| | E/GFGIDGPAIAKCLGE/K | K954 | 2 | 833.9 | 1.0963 | 0.0439 | | X |
| | E/GNKRITCRNGQWSEPPKCLHPCVISRE/I | K1130 | 5 | 689.13 | 0.5110 | 0.0738 | X | |
| Complement C3c α′ chain fragment | E/SPMYSIITPNILRLE/S | R35 | 2 | 955.5 | 0.1855 | 0.0148 | | X |
| | E/AHDAQGDVPVTVTVHDFPGKKLVLSSE/K | K43 | 5 | 602.51 | 0.4982 | 0.0454 | X | |
| | R/SVQLTEKRMDKVGKYPKELRKCCE/D | K660 | 3 | 1048.87 | 0.5759 | 0.1235 | X | |
| | R/SVQLTEKRMDKVGKYPKELRKCCE/D | K663 | 3 | 1048.87 | 0.5454 | 0.1140 | X | |
| | E/ACKKVFLDCCNYITE/L | K721 | 2 | 1042.46 | 0.4732 | 0.0451 | | X |
| Ig γ-4 chain C region | L/FPPKPKDTLMISRTPE/V | K126 | 4 | 734.89 | 0.7074 | 0.0589 | X | |
| Histidine-rich glycoprotein | E/SCPGKFKSGFPQVSMFFTHTFPK/- | K489 | 4 | 706.84 | 0.3392 | 0.0793 | X | |
| Ugl-Y3 | E/FKCDPHEATCYDDGKTYHVGE/Q | K1922 | 4 | 673.78 | 0.5242 | 0.0716 | X | |
| α-2-HS-glycoprotein chain B | E/TTCHVLDPTPVARCSVRQLKE/H | R81 | 3 | 877.11 | 0.7703 | 0.0188 | X | |
| | E/KQYGFCKATLSEKLGGAE/V | K207 | 2 | 1075.53 | 0.8623 | 0.1190 | | X |
| | E/FTVSGTDCVAKE/A | K211 | 2 | 738.33 | 0.8080 | 0.0803 | | X |
| | E/KQYGFCKATLSEKLGGAE/V | K213 | 3 | 717.02 | 0.5899 | 0.0474 | X | |
| Ig γ-2 chain C region | N/FGTQTYTCNVDHKPSNTKVDKTVE/R | K88 | 4 | 733.85 | 0.4808 | 0.0295 | X | |
| | E/RKCCVECPPCPAPPVAGPSVFLFPPKPKDTLMISRTPE/V | K125 | 5 | 900.45 | 0.2844 | 0.0173 | X | |
| Fibrinopeptide B | E/YCRTPCTVSCNIPVVSGKECEE/I | K195 | 3 | 936.4 | 0.3881 | 0.0301 | X | |
| Spectrin β chain, brain 1 | W/KSLLDACESRRVRLVD/T | R1893 | 3 | 693.7 | 1.2992 | 0.0484 | X | |
| Ig α-1 chain C region | E/SKTPLTATLSKSGNTFRPE/V | K212 | 3 | 733.05 | 0.3304 | 0.0207 | X | |
| | E/SKTPLTATLSKSGNTFRPE/V | K221 | 3 | 733.05 | 0.3304 | 0.0207 | X | |
| Fibrinopeptide A | D/LVPGNFKSQLQKVPPEWKALTDMPQMRME/L | K338 | 4 | 891.45 | 0.4513 | 0.0286 | X | |
| α-2-macroglobulin | ./SVSGKPQYMVLVPSLLHTE/T | K5 | 3 | 749.72 | 0.3098 | 0.0251 | X | |
| Afadin | E/KRMQEF/R | R232 | 3 | 499.29 | 0.2904 | 0.0779 | X | |
| Crooked neck-like protein 1 | K/EERLMLLE/S | R590 | 3 | 404.2 | 0.7511 | 0.1243 | X | |
| A-kinase anchor protein 9 | E/KETIIEELNTKIIEE/E | K349 | 2 | 980.03 | 0.4504 | 0.0297 | | X |
| Microtubule-actin | A/PDSQGKTDLTEIQCD/M | R2341 | 3 | 595.99 | 1.2817 | 0.0371 | | X |
| cross-linking factor 1 | D/IKARAEEREI/K | K3901 | 2 | 688.39 | 0.7046 | 0.1619 | | X |
| Plasmin light chain B | D/GKRAPWCHTTNSQVRWE/Y | K252/R253/R265 | 3 | 757.1 | 0.1447 | 0.0338 | | X |
| Myosin-15 | I/DIYLLEKSRVIFQQAGE/R | K283 o R285 | 2 | 1125.05 | 0.6073 | 0.0136 | | X |
| Hemopexin | E/KGYPKLLQDEFPGIPSPLDAAVE/C | K103 | 2 | 1324.18 | 0.6199 | 0.0675 | | X |
| Receptor-interacting serine/threonine-protein kinase 5 | E/LYESLMNIANRKQEE/M | K412 | 2 | 1008.45 | 0.5191 | 0.1124 | | X |
| Obscurin-like protein 1 | D/GGFVLKVLYCQAKD/R | K305 | 2 | 880.4 | 0.9896 | 0.0449 | | X |
| Myomegalin | D/LDTVAGLEKE/L | K753 | 2 | 576.29 | 0.3370 | 0.0897 | | X |
| UPF0639 protein | E/YLDKLMEETEE/L | K169 | 2 | 750.41 | 0.4540 | 0.0628 | | X |
| Ubiquitin carboxyl-terminal hydrolase 15 | D/KYQEELNFDNPLGMRGEI/A | K292 | 3 | 750.04 | 0.6775 | 0.0310 | | X |
| Rho GTPase-activating protein 10 | E/KFRKEQLGAVKEEKKK/F | K128 | 2 | 1054.47 | 0.4989 | 0.0137 | | X |
| Remodeling and spacing factor 1 | D/RWEKYLIKYLCE/C | R76 | 2 | 931.89 | 0.7567 | 0.0379 | | X |

TABLE 3
Glycated proteins identified in plasma analysis with the prediction approach.

| | | | | | Peak area | | |
|---|---|---|---|---|---|---|---|
| protein | peptide | gly site | charge | m/z 12Glu | ratio | SD | % increase |
| Serum albumin | I/AFAQYLQQCPFEDHVKLVNE/V | K65 | 3 | 867.08 | 3.4527 | 0.2841 | 82.25 |
| | E/VTEFAKTCVADESAE/N | K75 | 2 | 909.90 | 4.2010 | 0.1189 | 62.54 |
| | E/NCDKSLHTLFGDKLCTVATLRE/T | K88 | 4 | 685.84 | 1.7275 | 0.1213 | 280.38 |
| | E/SAENCDKSLHTLFGDKLCTVATLRE/T | K97 | 4 | 757.61 | 2.1800 | 0.0604 | 169.79 |
| | E/TYGEMADCCAKQEPERNE/C | K117 | 3 | 783.98 | 1.9826 | 0.1188 | 205.68 |
| | E/RNECFLQHKDDNPNLPRLVRPE/V | K130 | 5 | 582.69 | 1.8020 | 0.0553 | 250.15 |
| | E/CFLQHKDDNPNLPRLVRPE/V | R138 | 3 | 837.42 | 1.9867 | 0.0608 | 203.21 |
| | C/TAFHDNEETFLKKYLYE/I | K160 | 4 | 578.27 | 2.6194 | 0.1890 | 124.69 |
| | E/VDVMCTAFHDNEETFLKKYLYE/I | K161 | 3 | 977.78 | 2.6362 | 0.2000 | 123.51 |
| | E/LLFFAKRYKAAFTE/C | R184 | 2 | 934.50 | 2.5395 | 0.3140 | 134.13 |
| | E/CCQAADKAACLLPKLDE/L | K198 | 3 | 708.99 | 2.2734 | 0.0919 | 157.61 |
| | E/CCQAADKAACLLPKLDELRDEGKASSAKQRLKC | K205 | 6 | 803.07 | 1.8450 | 0.0341 | 236.94 |
| | ASLQKFGE/R | | | | | | |
| | E/LRDEGKASSAKQRLKCASLQKFG/E | K214 | 5 | 548.89 | 2.0810 | 0.1205 | 186.52 |
| | R/DEGKASSAKQRLKCASLQKFGE/R | K219 | 4 | 650.83 | 3.8603 | 0.2522 | 70.27 |
| | E/LRDEGKASSAKQRLKCASLQKFGE/R | R221 | 5 | 574.70 | 2.4759 | 0.0206 | 135.53 |
| | E/LRDEGKASSAKQRLKCASLQKFGE/R | K223 | 5 | 574.70 | 2.4759 | 0.0206 | 135.53 |
| | E/LRDEGKASSAKQRLKCASLQKFG/E | K229 | 5 | 548.89 | 2.0810 | 0.1205 | 186.52 |
| | E/LRDEGKASSAKQRLKCASLQKFGE/R | K219R221 | 5 | 607.11 | 5.9930 | 0.1294 | 40.07 |
| | E/LRDEGKASSAKQRLKCASLQKFGE/R | R221K223 | 5 | 607.11 | 5.9930 | 0.1294 | 40.07 |
| | E/LRDEGKASSAKQRLKCASLQKFGE/R | R221K229 | 5 | 607.11 | 5.9930 | 0.1294 | 40.07 |
| | E/RAFKAWAVARLSQRFPKAE/F | R242 | 4 | 479.67 | 1.0863 | 0.0165 | 2379.77 |
| | K/AWAVARLSQRFPKAE/F | K249 | 3 | 631.67 | 2.4648 | 0.2580 | 139.15 |
| | E/VSKLVTDLTKVHTE/C | K257 | 3 | 577.98 | 4.8835 | 0.0635 | 51.51 |
| | E/VSKLVTDLTKVHTECCHGDLLE/C | K264 | 3 | 906.11 | 4.5198 | 0.0700 | 56.84 |
| | E/CADDRADLAKYICE/N | K286 | 2 | 931.40 | 2.6254 | 0.0869 | 123.28 |
| | E/NQDSISSKLKE/C | K298 | 2 | 705.85 | 2.3950 | 0.0849 | 143.73 |
| | E/NQDSISSKLKECCEKPLLE/K | K300 | 3 | 814.06 | 2.1797 | 0.3488 | 182.11 |
| | E/NQDSISSKLKECCEKPLLE/K | K305 | 3 | 814.06 | 2.1797 | 0.3488 | 182.11 |
| | E/AKDVFLGMFLYE/Y | K347 | 2 | 805.89 | 2.7657 | 0.1946 | 114.25 |
| | E/KCCAAADPHECYAKVFDE/F | K383 | 3 | 778.32 | 3.3029 | 0.0262 | 86.85 |
| | D/PHECYAKVFDE/F | K396 | 3 | 778.32 | 3.3440 | 0.0298 | 85.33 |
| | E/FKPLVEEPQNLIKQNCE/L | K402 | 3 | 750.04 | 3.2217 | 0.0305 | 90.03 |
| | E/FKPLVEEPQNLIKQNCE/L | K413 | 3 | 750.04 | 3.2217 | 0.0305 | 90.03 |
| | E/LFEQLGEYKFQ/N | K426 | 2 | 782.38 | 2.7954 | 0.0977 | 111.62 |
| | Q/NALLVRYTKKVPQVSTPTLVE/V | R434 | 2 | 1260.21 | 2.3466 | 0.1925 | 150.43 |
| | R/YTKKVPQVSTPTLVE/V | K437 | 2 | 927.00 | 3.8202 | 0.3883 | 71.82 |
| | Q/NALLVRYTKKVPQVSTPTLVE/V | K438 | 2 | 1260.21 | 2.3466 | 0.1925 | 150.43 |
| | E/VSRNLGKVGSKCCKHPE/A | K456 | 4 | 530.26 | 3.4084 | 0.3502 | 84.32 |
| | E/VSRNLGKVGSKCCKHPE/A | K460 | 4 | 530.26 | 3.4084 | 0.3502 | 84.32 |
| | E/VSRNLGKVGSKCCKHPE/A | K463 | 4 | 530.26 | 3.4084 | 0.3502 | 84.32 |
| | E/KTPVSDRVTKCCTE/S | K499 | 3 | 614.96 | 3.8699 | 0.0948 | 69.74 |
| | E/SLVNRRPCFSALE/V | R508/R509 | 2 | 855.93 | 1.7271 | 0.0533 | 276.02 |
| | E/SLVNRRPCFSALEVDETYVPKEFNAE/T | K524 | 3 | 1078.19 | 1.9065 | 0.2118 | 229.47 |
| | E/TFTFHADICTLSEKE/R | K543 | 3 | 654.30 | 1.6370 | 0.0656 | 316.19 |
| | E/RQIKKQTALVE/L | K549 | 3 | 492.62 | 6.5192 | 0.0130 | 36.24 |
| | E/GKKLVAASQAALGL/- | K597 | 3 | 744.94 | 3.6728 | 0.0155 | 74.83 |
| Serotransferrin | A/VPDKTVRWCAVSE/H | K23 | 2 | 854.91 | 2.0552 | 0.2725 | 95.01 |
| | D/AYLAPNNLKPVVAE/F | K97 | 2 | 830.94 | 3.8603 | 0.1986 | 70.16 |
| | E/FYGSKEDPQTFYYAVAVVKKD/S | K121 | 3 | 873.43 | 1.7841 | 0.0492 | 255.75 |
| | E/FYGSKEDPQTFYYAVAVVKKD/S | K122 | 3 | 873.43 | 1.7841 | 0.0492 | 255.75 |
| | P/QTFYYAVAVVKKD/S | K121K122 | 2 | 873.43 | 3.0817 | 0.4106 | 98.95 |
| | N/IPIGLLYCDLPEPRKPLE/K | K162 | 3 | 763.19 | 2.1103 | 0.0877 | 180.85 |
| | K/DGAGDVAFVKHSTIFE/N | K225 | 2 | 927.94 | 3.0195 | 0.2601 | 100.07 |
| | E/YKDCHLAQVPSHTVVARSMGGKEDLIWE/L | K258 | 5 | 678.53 | 1.3617 | 0.0181 | 553.80 |
| | E/YKDCHLAQVPSHTVVARSMGGKE/D | R273 | 3 | 911.78 | 1.0741 | 0.1445 | 965.90 |
| | E/FQLFSSPHGKDLLFK/D | K315 | 3 | 643.01 | 3.0271 | 0.1654 | 99.12 |
| | E/WSVNSVGKIE/C | K384 | 2 | 640.82 | 2.1554 | 0.0996 | 173.94 |
| | E/FFSEGCAPGSKKDSSLCKLC/M | K508 | 3 | 814.37 | 3.7103 | 0.0528 | 73.81 |
| | E/FFSEGCAPGSKKDSSLCKLC/M | K509 | 4 | 657.79 | 1.9429 | 0.1067 | 213.93 |
| | E/KGDVAFVKHQTVPQNTGGKNPDPWAKNLN/E | K564 | 5 | 665.34 | 1.9385 | 0.0565 | 213.60 |
| | N/PDPWAKNLNEKDYE/L | K571 | 3 | 627.62 | 2.7389 | 0.1073 | 115.31 |
| | E/LLCLDGTRKPVEE/Y | R587 | 2 | 846.43 | 2.6776 | 0.1856 | 120.24 |
| | E/LLCLDGTRKPVEE/Y | K588 | 2 | 846.43 | 2.6776 | 0.1856 | 120.24 |
| | E/KYLGEEYVKAVGNLRKCSTSSLLE/A | K668 | 3 | 967.47 | 1.9425 | 0.2318 | 222.38 |
| | E/YVKAVGNLRKCSTSSLLE/A | K676 | 2 | 1094.07 | 1.8228 | 0.0455 | 243.56 |
| Titin | R/EVSRKTWTKVMD/F | K14587 | 7 | 547.94 | 3.0812 | 0.0772 | 96.18 |
| Haptoglobin β chain | E/HSVRYQCKNYYKLRTE/G | R49 | 4 | 537.02 | 4.6194 | 0.4786 | 55.93 |
| | E/GDGVYTLNDKKQWINKAVGDKLPE/C | K76 | 3 | 951.16 | 2.7233 | 0.2251 | 117.46 |
| | K/KQWINKAVGDKLPE/C | K77 | 3 | 596.65 | 2.7970 | 0.0937 | 111.50 |
| | K/KQWINKAVGDKLPECE/A | K82 | 3 | 693.01 | 2.6014 | 0.0305 | 124.92 |
| | E/AVCGKPKNPANPVQR/I | K151 | 4 | 450.24 | 4.5284 | 0.2733 | 56.91 |
| | E/RVMPICLPSKDYAE/V | K270 | 3 | 614.30 | 2.1056 | 0.0824 | 181.59 |
| | E/KKTPKSPVGVQPILNE/H | K321 | 2 | 949.04 | 3.0825 | 0.1091 | 96.22 |
| | E/GSTVPEKKTPKSPVGVQPILNE/H | K322 | 2 | 1234.67 | 3.0408 | 0.1829 | 98.54 |
| Apolipoprotein A-I | ./DEPPQSPWDRVKD/L | K12 | 3 | 563.65 | 4.2997 | 0.4251 | 61.31 |
| | E/FWDNLEKETE/G | K77 | 2 | 736.83 | 2.9621 | 0.2513 | 103.03 |
| | E/VKAKVQPYLDDFQKKWQEE/M | K94 | 3 | 847.77 | 1.3809 | 0.0311 | 527.26 |
| | E/VKAKVQPYLDDFQKKWQEE/M | K96 | 3 | 847.77 | 1.3809 | 0.0311 | 527.26 |
| | E/MELYRQKVEPLRAE/L | R116 | 3 | 642.00 | 3.0683 | 0.2103 | 97.34 |
| | E/LYRQKVEPLRAELQE/G | K118 | 3 | 678.70 | 2.6561 | 0.0556 | 120.86 |
| | E/YHAKATEHLSTLSEKAKPALE/D | K195 | 4 | 622.32 | 2.4860 | 0.1557 | 135.53 |
| Haptoglobin-related protein | K/KQWINKAVGDKLPE/C | K77 | 3 | 596.65 | 2.7970 | 0.0937 | 111.50 |
| | K/KQWINKAVGDKLPECE/A | K82 | 3 | 693.01 | 2.6014 | 0.0305 | 124.92 |
| | E/AVCGKPKNPANPVQR/I | K151 | 4 | 450.24 | 4.5284 | 0.2733 | 56.91 |
| Plasmin light chain B | E/LCDIPRCTTPPPSSGPTYQCLKGTGE/N | K258 | 3 | 1018.80 | 2.6289 | 0.0613 | 122.89 |
| Ig γ-4 chain C region | L/FPPKPKDTLMISRTPE/V | K126 | 3 | 673.69 | 1.9653 | 0.2180 | 213.62 |
| α-1-antitrypsin | E/GLKLVDKFLE/D | K153 | 2 | 662.37 | 2.5551 | 0.2162 | 130.18 |
| | E/LTHDIITKFLE/N | K298 | 3 | 497.93 | 3.5686 | 0.1205 | 77.98 |
| | G/KLQHLENELTHDIITKFLE/N | K283K298 | 2 | 867.95 | 3.0064 | 0.4063 | 102.18 |
| | E/EAPLKLSKAVHKAVLTIDE/K | K352 | 2 | 1113.13 | 2.0188 | 0.1569 | 199.69 |
| | E/EAPLKLSKAVHKAVLTIDEKGTE/A | K355 | 4 | 660.62 | 2.9440 | 0.0554 | 102.94 |
| | E/EAPLKLSKAVHKAVLTIDEKGTE/A | K359 | 4 | 660.62 | 2.9440 | 0.0554 | 102.94 |
| | N/TKSPLFMGKVVNPTQK/- | K404 | 3 | 646.35 | 2.9835 | 0.3288 | 102.85 |
| | E/QNTKSPLFMGKVVNPTQK/- | K418 | 3 | 727.05 | 2.2207 | 0.3217 | 171.33 |
| Girdin (APE) (HkRP1) | S/EVSRYKE/R | R331K333 | 3 | 412.19 | 2.8321 | 0.1307 | 109.53 |
| α-2-HS-glycoprotein chain B | E/VKVWPQQPSGELFE/I | K49 | 2 | 903.45 | 2.9152 | 0.3063 | 106.42 |
| | E/TTCHVLDPTPVARCSV/R | R81 | 3 | 658.98 | 2.4859 | 0.1301 | 202.28 |
| | E/FTVSGTDCVAKEATE/A | K193 | 3 | 888.90 | 2.0658 | 0.1211 | 189.32 |
| | E/AAKCNLLAEKQYGFCKATLSE/K | K207 | 2 | 855.93 | 1.7500 | 0.0651 | 267.99 |
| | G/FCKATLSEKLGGAE/V | K213 | 3 | 558.28 | 1.7131 | 0.2072 | 295.10 |
| | E/KQYGFCKATLSEKLGGAE/V | K219 | 3 | 717.02 | 3.8009 | 0.2399 | 71.77 |
| Centriolin | H/ERARRLMKE/L | R2115/K2118 | 3 | 510.26 | 4.4383 | 0.3934 | 58.71 |
| Complement factor H | E/VVKCLPVTAPENGKIVSSAMEPDRE/Y | K127 | 3 | 961.82 | 2.9292 | 0.4225 | 106.86 |
| | E/MHCSDDGFWSKEKPKCVE/I | K200 | 4 | 601.26 | 4.0614 | 0.1642 | 65.46 |
| | E/ISCKSPDVINGSPISQKIIYKENE/R | K193 o K206 | 3 | 961.82 | 1.8912 | 0.2486 | 235.42 |
| | E/TTCYMGKWSSPPQCE/G | K501 | 2 | 997.90 | 2.3201 | 0.2439 | 154.88 |
| Ig γ-1 chain C region | E/LLGGPSVFLFPPKPKDTLMISRTPE/V | K129 | 3 | 673.69 | 1.9907 | 0.1311 | 204.46 |
| | P/KDTLMISRTPE/V | K131 | 3 | 674.02 | 2.4707 | 0.1130 | 136.52 |
| β-2-glycoprotein 1 | ./GRTCPKPDDLPFSTVVPLKTFYEPGEE/I | K19 | 4 | 811.14 | 1.4574 | 0.1616 | 486.61 |
| | E/KFKNGMLHGDKVSFFCKNKE/K | K276 | 3 | 860.09 | 1.2985 | 0.0620 | 688.00 |
| | E/HSSLAFWKTDASDVKPC/- | K317 | 3 | 704.33 | 2.7325 | 0.0760 | 115.59 |
| Ig γ-2 chain C region | N/FGTQTYTCNVDHKPSNTKVDKTVE/R | K88 | 4 | 733.85 | 4.188749987 | 0.443489 | 63.57 |
| | E/RKCCVECPPCPAPPVAGPSVFLFPPKPKDTLMI | K125 | 5 | 900.45 | 1.1342 | 0.0396 | 1570.34 |
| | SRTPE/V | | | | | | |
| α-2-macroglobulin | ./SVSGKPQYMVLVPSLLHTETTE/K | K5 | 3 | 860.10 | 4.5263 | 0.1135 | 56.76 |
| | E/GLRVGFYE/S | K681 | 2 | 551.77 | 1.7237 | 0.1118 | 281.03 |
| Nesprin-1 | E/SLDKLSQR/G | K2583/R2587 | 2 | 635.82 | 5.8942 | 0.5242 | 41.17 |
| Centrosome-associated protein | G/ERELLQAAKE/N | K1294 | 3 | 450.24 | 2.9619 | 0.1153 | 102.17 |
| Coiled-coil domain-containing protein 135 | R/EEEERLMEAEKAKPD/A | K225 | 3 | 655.34 | 3.1375 | 0.3244 | 94.94 |
| Nucleoprotein TPR | R/SQNTKISTQLDFASKRYE/M | K722/R723 | 4 | 610.79 | 1.6746 | 0.0539 | 297.69 |
| Ig κ chain C region | E/AKVQWKVDNALQSGNSQESVTE/Q | K37 | 2 | 1291.62 | 1.0368 | 0.0362 | 22644.48 |
| | Y/EKHKVYACEVTHQGLSSPVTKSFNRGEC/- | K80 | 3 | 1134.04 | 2.5931 | 0.0997 | 125.88 |
| | E/KHKVYACEVTHQGLSSPVTKSFNRGEC/- | K80/K82 | 2 | 1133.54 | 2.4075 | 0.0990 | 142.55 |
| | E/VTHQGLSSPVTKSFNRGEC/- | K99 | 3 | 756.03 | 1.5876 | 0.0264 | 340.82 |
| Nebulette | D/AAYKGVHPHIVEMDRRPGII/V | K822 | 5 | 485.06 | 1.9903 | 0.1493 | 205.26 |
| Ig α-1 chain C region | E/SKTPLTATLSKSGNTFRPE/V | K212 | 3 | 733.05 | 2.8178 | 0.1237 | 110.36 |
| Histidine-rich glycoprotein | E/SCPGKFKSGFPQVSMFFTHTFPK/- | K489 | 4 | 706.84 | 1.1471 | 0.0695 | 1558.36 |
| Fibrinopeptide A | E/SSSHHPGIAEFPSRGKSSSYSKQFTSSTSYNRG | R573 o K581 | 5 | 861.39 | 2.4299 | 0.0402 | 139.94 |
| | DSTFE/S | | | | | | |
| Fibrinopeptide B | E/RKAPDAGGCLHADPDLGVLCPTGCQLQE/A | K44 | 3 | 539.25 | 1.9038 | 0.1289 | 224.61 |
| | E/YCRTPCTVSCNIPVVSGKECEE/I | K195 | 3 | 936.40 | 3.1492 | 0.1627 | 93.42 |
| | E/MEDWKGDKVKAHYGGFTVQNE/A | K304 | 3 | 868.07 | 1.7966 | 0.2224 | 267.22 |
| Apolipoprotein A-II | E/AKSYFEKSKEQLTPLIKKAGTE/L | K44 | 5 | 532.49 | 2.6724 | 0.0739 | 119.74 |
| | E/AKSYFEKSKEQLTPLIKKAGTE/L | K46 | 5 | 532.49 | 2.6724 | 0.0739 | 119.74 |
| Serine/threonine-protein phosphatase | R/EKKKELEREE/L | K447 o R451 | 3 | 547.95 | 3.2961 | 0.2921 | 88.02 |
| 2A 56 kDa regulatory subunit α | | | | | | | |
| TBC1 domain family member 1 | E/EVQKLRPRNEQRENE/L | R437 o R439 | 3 | 750.37 | 2.8718 | 0.0334 | 106.87 |
| Protein max | F/QSAADKRAHHNALERKRRD/H | K24 | 5 | 485.06 | 1.9495 | 0.1023 | 212.19 |
| Vitamin D-binding protein | E/ACCAEGADPDCYDTRTSALSAKSCE/S | K78 | 3 | 986.72 | 2.9824 | 0.0682 | 100.97 |
| | E/RKLCMAALKHQPQEFPTYVEPTNDEICE/A | R103 | 3 | 1190.23 | 2.0961 | 0.2027 | 186.32 |
| | R/KLCMAALKHQPQEFPTYVEPTNDEICE/A | K104 | 3 | 1189.89 | 2.0961 | 0.2027 | 186.32 |
| Complement C3c α′ chain | ./SPMYSIITPNILRLE/S | Nterm | 2 | 955.51 | 1.2567 | 0.0463 | 798.20 |
| | E/ACKKVFLDCCNYITE/L | K699 | 2 | 1041.96 | 3.1165 | 0.2680 | 95.51 |
| | E/KEDGKLNKLCRD/E | K1475/R1485 | 2 | 879.48 | 4.1808 | 0.0686 | 62.90 |
| Apolipoprotein C-I | P/DVSSALDKLKE/F | K10 | 2 | 683.86 | 3.5058 | 0.0337 | 79.82 |
| | ./TPDVSSALDKLKE/F | K12 | 2 | 782.90 | 0.9851 | 0.0962 | −7153.82 |
| Low molecular weight growth-promoting | E/ATKTVGSDTFYSFKYE/I | K46 o K57 | 2 | 1003.47 | 2.6208 | 0.2090 | 124.09 |
| factor | E/IKEGDCPVQSGKTWQDCE/Y | K71 | 3 | 767.33 | 2.4972 | 0.2027 | 135.11 |
| Fibrinogen γ chain | ./YVATRDNCCILDERFG/S | R5 o R14 | 3 | 767.33 | 2.4972 | 0.2027 | 135.11 |
| | E/IYNSNNQKIVNLKE/K | K120 | 2 | 919.98 | 4.3279 | 0.4520 | 60.47 |
| Vitamin K-dependent protein S | E/GYRYNLKSKSCEDIDECSE/N | K196 | 3 | 839.36 | 2.9659 | 0.2260 | 102.68 |
| | E/TKVYFAGFPRKVE/S | K383 | 3 | 568.64 | 1.2968 | 0.0574 | 720.41 |
| Apolipoprotein C-II | ./TQQPQQDEMPSPTFLTQVKE/S | K19 | 2 | 1248.09 | 2.3605 | 0.1958 | 148.20 |
| Apolipoprotein D | E/IEKIPTTFE/N | K31 | 3 | 620.32 | 1.2724 | 0.1010 | 1273.73 |
| Complement C1s subcomponent light chain | E/VLGPELPKCVPVCGVPREPFEE/K | K405 | 3 | 891.11 | 1.5555 | 0.0316 | 360.64 |
| Golgin subfamily A member 3 | E/QVRLQARKWLEEQLKQYRVKRQQ/E | R166/K168 | 2 | 1125.56 | 3.6073 | 0.1217 | 76.83 |
| DnaJ homolog subfamily C member 13 | E/HRTELLTEALRFRTD/F | R90 | 2 | 1009.98 | 1.4006 | 0.0725 | 517.62 |
| Hemopexin | E/FVWKSHKVVDRELISE/R | K54 | 3 | 708.36 | 1.5739 | 0.1749 | 386.46 |
| Receptor-interacting serine/threonine- | E/LYESLMNIANRKQEE/M | R412 | 2 | 1007.95 | 2.2719 | 0.0170 | 157.44 |
| protein kinase 5 | | | | | | | |
| Apolipoprotein A-IV | E/LTQQLNALFQDKLGE/V | K45 | 2 | 940.49 | 3.3190 | 0.4918 | 90.58 |
| WW domain-binding protein 11 | K/MKDPKQIIRDME/K | K48 | 2 | 849.43 | 2.0001 | 0.2673 | 210.25 |
| Complement C4 γ chain | E/VKKYVLPNFE/V | K215 | 2 | 699.88 | 1.1009 | 0.0629 | 2757.35 |
| Obscurin-like protein 1 | D/GGFVLKVLYCQAKD/R | K305 | 2 | 880.40 | 4.1786 | 0.0929 | 62.98 |
| Transmembrane and TPR repeat-containing | E/LKALPILEELLRYYPD/H | K720 | 2 | 1054.98 | 2.6859 | 0.0404 | 118.68 |
| protein 3 | | | | | | | |
| Putative alpha-1-antitrypsin-related | E/YITNFPLFIGKVVNPTQK/- | K392 o K399 | 3 | 721.71 | 1.7335 | 0.0128 | 273.96 |
| protein | | | | | | | |

### REFERENCES

1. Costello, C. E., Contado-Miller, J. M., Cipollo, J. F. (2007) A glycomics platform for the analysis of permethylated oligosaccharide alditols. J. Am. Soc. Mass Spectrom. 18, 1799-1812.
2. Maillard, L. C. (1912) Action des acides amines sur le sucres: formation des mélanoidines per voie méthodique. C. R. Acad. Sci. 154, 66-68.
3. Thornalley, P. J., Langborg, A., Minhas, H. S. (1999) Formation of glyoxal, methylglyoxal and 3-deoxyglucosone in the glycation of proteins by glucose. Biochem. J. 344, 109-116.
4. Ulrich, P., Cerami, A. (2001) Protein glycation, diabetes, and aging. Recent Progress Hormone Res. 56, 1-22.
5. Brock, J. W. C., Hinton, D. J. S., Cotham, W. E., Metz, T. O., Thorpe, S. R., Baynes, J. W., Ames, J. M. (2003) Proteomic analysis of the site specificity of glycation and carboxymethylation of ribonuclease. J. Proteome Res. 2, 506-513.
6. Brunner, Y., Schvartz, D., Priego-Capote, F., Couté, Y., Sanchez, J. C. (2009) Glucotoxicity and pancreatic proteomics. J. Proteomics. 71, 576-591.
7. Reusch, J. E. (2003) Diabetes, microvascular complications, and cardiovascular complications: what is it about glucose? J. Clin. Invest. 112, 986-988.
8. LeRoith, D. (2002) Beta-cell dysfunction and insulin resistance in type 2 diabetes: role of metabolic and genetic abnormalities. Am. J. Med. 113 Suppl. 6A, 3S-11S.
9. Dubois, M., Vacher, P., Roger, B., Huyghe, D., Vandewalle, B., Kerr-Conte, J., Pattou, F., Moustaid-Moussa, N., Lang, J. C. (2007) Glucotoxicity inhibits late steps of insulin exocytosis. Endocrinology 148, 1605-1614.
10. Baynes, J. W. (2001) The role of AGEs in aging: causation or correlation. Exp. Gerontol. 36, 1527-1537.
11. Brownlee, M. (2001) Biochemistry and molecular cell biology of diabetic complications. Nature 14, 813-820.
12. Hipkiss, A. R. (2006) Accumulation of altered proteins and ageing: causes and effects. Exper. Gerontol. 41, 464-473.
13. Calvo, C., Talussot, C., Ponsin, G., Berthezene, F. (1988) Nonenzymatic glycation of apolipoprotein A-I. Effects on its self-association and lipid binding properties. Biochem. Biophys. Res. Comm. 153, 1060-1067.
14. Calvo, C., Verdugo, C. (1992) Association in vivo of glycated apolipoprotein A-I with high density lipoproteins. Eur. J. Clin. Chem. Clin. Biochem. 30, 3-5.
15. Calvo, C., Ponsin, G., Berthezene, F. (1998) Characterization of the nonenzymatic glycation of high-density lipoprotein in diabetic patients. Diabete Metab. 14, 264-269.
16. Abdel-Wahab, Y. H. A., O'Harte, F. P. M., Boyd, A. C., Barnett, C. R., Flatt, P. R. (1997) Glycation of insulin results in reduced biological activity in mice. Acta Diabetol. 34, 265-270.
17. Miedema, K. (2004) Towards worldwide standardisation of HbA₁c determination. Diabetologia 47, 1143-1148.
18. Ladyzynski, P., Wojcicki, J. M., Bak, M., Sabalinska, S., Kawiak, J., Foltynski, P., Krzymien, J., Karnafel, W. (2008) Validation of haemoglobin glycation models using glycemia monitoring in vivo and culturing of erythrocytes in vitro. Ann. Biomed. Eng. 36, 1188-1202.
19. Saudek, C. D., Derr, R. L., Kalyani, R. R. (2006) Assessing glycemica in diabetes using self-monitoring blood glucose and hemoglobin A1c. J. Am. Med. Assoc. 295, 1688-1697.
20. Brownlee, M., Hirsch, I. B. (2006) Glycemic variability: a hemoglobin A1c-independent risk factor for diabetic complications. J. Am. Med. Assoc. 295, 1707-1708.
21. Kisugi, R., Kouzuma, T., Yamamoto, T., Akizuki, S., Miyamoto, H., Someya, Y., Yokoyama, J., Abe, I., Hirai, N., Ohnishi, A. (2007) Structural and glycation site changes of albumin in diabetic patient with very high glycated albumin. Clin. Chim. Acta 382, 59-64.
22. Yoshiuchi, K., Matsuhisa, M., Katakami, N., Nakatani, Y., Sakamoto, K., Matsuoka, T., Umayahara, Y., Kosugi, K., Kaneto, H., Yamakasi, Y., Hori, M. (2008) Glycated albumin is a better indicator for glucose excursion than glycated hemoglobin in type 1 and type 2 diabetes. Endocrine J. 55, 503-507.
23. Zhang, Q., Tang, N., Schepmoes, A. A., Phillips, L. S., Smith, R. D., Metz, T. O. (2008) Proteomic profiling of nonenzymatically glycated proteins in human plasma and erythrocyte membranes. J. Proteome Res. 7, 2025-2032.
24. Zhang, Q., Petyuk, V. A., Schepmoes, A. A., Orton, D. J., Monroe, M. E., Feng, Y., Smith, R. D., Metz, T. O. (2008) Analysis of non-enzymatically glycated peptides: neutral-loss-triggered MS3 versus multi-stage activation of tandem mass spectrometry. Rap. Comm. Mass Spectrom. 22, 3027-3034.
25. Zhang, Q., Ames, J. M., Smith, R. D., Baynes, J. W., Metz T. O. (2009) A perspective on the Maillard reaction and the analysis of protein glycation by mass spectrometry: probing the pathogenesis of chronic disease. J. Proteome Res. 8, 754-769.
26. Ref. Phenyx.
27. Ref. SuperHirn.
28. Hoopmann, M. R., Finney, G. L., MacCoss, M. J. (2007) High speed data reduction, feature selection, and MS/MS spectrum quality assessment of shotgun proteomics datasets using high resolution mass spectrometry. Anal. Chem. 79, 5630-5632.
29. Elias, J. E., Gygi, S. P. (2007) Target-decoy search strategy for increased confidence in large-scale protein identifications by mass spectrometry. Nat. Methods 4, 207-214.
30. Mueller, L. N., Rinner, O., Schmidt, A., Letarte, S., Bodenmiller, B., Brusniak, M., Vitek, O., Aebersold, R., Müller, M. (2007). SuperHirn—a novel tool for high resolution LC-MS-based peptide/protein profiling. Proteomics 7, 3470-3480.
31. Pedrioli, P. G., Eng, J. K., Hubley, R., Vogelzang, M., Deutsch, E. W., Raught, B., Pratt, B., Nilsson, E., Angeletti, R. H., Apweiler, R., Cheung, K., Costello, C. E., Hermjakob, H., Huang, S., Julian, R. K., Kapp, E., McComb, M. E., Oliver, S. G., Omenn, G., Paton, N. W., Simpson, R., Smith, R., Taylor, C. F., Zhu, W., Aebersold, R. (2004). A common open representation of mass spectrometry data and its application to proteomics research. Nat Biotechnol. 22, 1459-1566.
32. Tusher, V. G., Tibshirani, R., Chu G. (2001). Significance analysis of microarrays applied to the ionizing radiation response. Proc. Natl. Acad. Sci. USA. 98, 5116-5121.
33. Efron, B., Tibshirani, R., Storey, J. D., Tusher, V. (2001). Empirical Bayes analysis of a microarray experiment. J. Am. Stat. Soc. 96, 1151-1160.
34. Macek, B., Mann, M., Olsen, J. V. (2009). Global and site-specific quantitative phosphoproteomics: principles and applications. Annu. Rev. Pharmacol. Toxicol. 49, 199-221.
35. Jeric, I., Versluis, C., Horvat, S., Heck, A. J. R. (2002) Tracing glycoprotein structures: electron ionization tandem mass spectrometric analysis of sugar-peptide adducts. J. Mass Spectrom. 37, 803-811.
36. Frolov, A., Hoffmann, P., Hoffmann, R. (2006) Fragmentation behaviour of glycated peptides derived from D-glucose, D-fructose and D-ribose in tandem mass spectrometry. J. Mass Spectrom. 41, 1459-1469.
37. Scherl, A., Shannon Tsai, Y., Shaffer, S. A., Goodlett, D. R. (2008) Increasing information from shotgun proteomics data by accounting for misassigned precursor ion masses. Proteomics 8, 2791-2797.
38. Scherl, A., Shaffer, S. A., Taylor, G. K., Hernandez, P., Appel, R. D., Binz, P. A., Goodlett, D. R. (2008) On the benefits of acquiring peptide fragment ions at high measured mass accuracy. J. Am. Soc. Mass Spectrom. 19, 891-901.
39. Olsen, J. V., Macek, B., Lange, O., Makarov, A., Horning, S., Mann, M. (2007) Higher-energy C-trap dissociation (HCD) for precise peptide modification analysis. Nat. Methods 4, 709-712.
40. Mann, M., Kelleher, N. L. (2008) Precision proteomics: the case for high resolution and high mass accuracy. PNAS 105, 18132-18138.
41. Stanaway, S. E., Gill, G. V. (2000) Protein glycosylation in diabetes mellitus: biochemical and clinical considerations. Pract. Diab. Int. 17, 21-25.
42. Saynes, K. C. R., Mc Intosh, C., Feher, M. D. (2001) Artifactually low glycated hemoglobin is a potential pitfall in diabetes management: consider congenital hemolytic anemias. Pract. Diab. Int. 18, 103-106.
43. Chandalia, H. B., Krishnaswamy, P. R. (2002) Glycated hemoglobin. Curr. Sci. 83, 1522-1532.
44. Shilton, B. H., Campbell, R. L., Walton, D. J. (1993) Site specificity of glycation of horse liver alcohol dehydrogenase in vitro. Eur. J. Biochem. 215, 567-572.
45. Iberg, N., Flückiger, R. (1986) Nonenzymatic glycosylation of albumin in vivo. Identification of multiple glycosylated sites. J. Biol. Chem. 261, 13542-13545.
46. Acosta, J., Hetting a, J., Flückiger, R., Krumrei, N., Goldfine, A., Angarita, L., Halperin, J. (2000) Molecular basis for a link between complement and the vascular complications of diabetes. Prog. Natl. Acad. Sci. U.S.A 97, 5450-5455.
47. Baynes, J. W., Watkins, N. G., Fisher, C. I., Hull, C. J., Patrick, J. S., Ahmed, M. U., Dunn, J. A., Thorpe, S. R. (1989) The Amadori product on protein: structure and reactions. Prog. Clin. Biol. Res. 340, 783-795.
48. Johansen, M. B., Kiemer, L., Brunak, S. (2006) Analysis and prediction of mammalian protein glycation. Glycobiology 16, 844-853.

1-13. (canceled)

14. A method for analysis of one or more glycated proteins in a sample, the glycated proteins containing moieties of a natural reducing carbohydrate bound at one or more glycation sites in the proteins, the method comprising:
treating the sample with a stable isotopic form of said carbohydrate which is different in mass from the natural carbohydrate, whereby the isotopic form becomes incorporated by glycation in one or more proteins in the sample, and one or more of said proteins are accordingly glycated by the natural reducing carbohydrate and by the isotopic form of the carbohydrate at identical glycation sites; and
identifying and/or quantifying the glycated proteins by the difference in mass between the natural carbohydrate and the isotopic form of the carbohydrate at identical glycation sites.

15. The method according to claim 14, in which the natural reducing carbohydrate is selected from glucose, fructose, ribose, mannose, ascorbic acid, glyoxal or methylglyoxal.

16. The method according to claim 15, in which the natural reducing carbohydrate is glucose.

17. The method according to claim 14, in which the isotopic form of the carbohydrate is the ¹³C isotope, the ²H isotope or the ¹⁸O isotope.

18. The method according to claim 16, in which the natural reducing carbohydrate ¹²C6-glucose and the isotopic form is ¹³C6-glucose.

19. The method according to claim 14, in which the proteins are digested to form peptides by treatment with an endoproteinase.

20. The method according to claim 14, in which the digestion step is carried out with Glu-C, trypsin, Asp-N, Arg-C, or CNBr.

21. The method according to claim 14, in which the glycated proteins or peptides are identified and/or quantified by mass spectrometry, wherein doublet signals are obtained, corresponding to each glycation site, with a mass shift corresponding to the difference in mass between the natural carbohydrate and the isotopic carbohydrate.

22. The method according to claim 21, in which the glycated proteins or peptides are quantified by measuring the signal intensity corresponding to glycation with the natural carbohydrate and comparing it to the signal intensity corresponding to glycation with a predetermined quantity of the isotopic form of the carbohydrate at the same glycation site.

23. The method according to claim 21, in which tandem mass spectrometry is carried out to identify and/or quantify the glycated peptides, and hence identify proteins from which the glycated peptides have been derived and the glycation sites for a specific protein in the sample, and optionally to quantify the degree of glycation at such sites.

24. The method according to claim 21, in which the glycated peptides are fractionated by reversed-phase liquid chromatography prior to analysis by mass spectrometry.

25. A method for analysis of one or more glycated proteins in a sample, the glycated proteins containing moieties of a natural reducing carbohydrate bound at one or more glycation sites in the proteins, the method comprising:
a) treating the sample with a stable isotopic form of said carbohydrate which is different in mass from the natural carbohydrate, whereby the isotopic form becomes incorporated by glycation in one or more proteins in the sample;
b) digesting the proteins in the thus-treated sample to form peptides, at least some of which are glycated by the natural reducing carbohydrate and some by the isotopic form of the carbohydrate at identical glycation sites;
c) separating the glycated peptides from the non-glycated peptides; and
d) identifying and/or quantifying the glycated peptides by the difference in mass between the natural carbohydrate and the isotopic form of the carbohydrate at identical glycation sites.

26. The method according to claim 25, in which the natural reducing carbohydrate is selected from glucose, fructose, ribose, mannose, ascorbic acid, glyoxal or methylglyoxal.

27. The method according to claim 26, in which the natural reducing carbohydrate is glucose.

28. The method according to claim 25, in which the isotopic form of the carbohydrate is the ¹³C isotope, the ²H isotope or the ¹⁸O isotope.

29. The method according to claim 25, in which the natural reducing carbohydrate is ¹²C6-glucose and the isotopic form is ¹³C6-glucose.

30. The method according to claim 25, in which the proteins are digested to form peptides by treatment with an endoproteinase.

31. The method according to claim 25, in which the glycated peptides are separated from the non-glycated peptides by boronate affinity chromatography, cationic exchange chromatography, isoelectric focusing or reverse phase HPLC.

32. The method according to claim 25, in which the glycated proteins or peptides are identified and/or quantified by:
a) mass spectrometry, wherein doublet signals are obtained, corresponding to each glycation site, with a mass shift corresponding to the difference in mass between the natural carbohydrate and the isotopic carbohydrate;
b) measuring the signal intensity corresponding to glycation with the natural carbohydrate and comparing it to the signal intensity corresponding to glycation with a predetermined quantity of the isotopic form of the carbohydrate at the same glycation site; or
c) tandem mass spectrometry to identify and/or quantify the glycated peptides, and hence identify proteins from which the glycated peptides have been derived and the glycation sites for a specific protein in the sample, and optionally to quantify the degree of glycation at such sites.

33. The method according to claim 32, in which the glycated peptides are fractionated by reversed-phase liquid chromatography prior to analysis by mass spectrometry.
